SEQUENCE LISTING (27 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - DNA, 741 bp, Artificial Sequence
  source: /note=“Description of Artificial Sequence Synthetic polynucleotide”
  CDS (1)..(738)
gatatccagatgacccagtccccgtcctccctgtccgcctctgtgggcgatagggtcaccatcacctgcggcgccagcgaaaacatctatggcgcgctgaactggtatcaacagaaacccgggaaagctccgaagcttctgatttacggtgcgacgaacctggcagatggagtcccttctcgcttctctggatccggctccggaacggatttcactctgaccatcagcagtctgcagcctgaagacttcgctacgtattactgtcagaacgttttaaatactccgttgactttcggacagggtaccaaggtggaaataaaacgtactggcggtggtggttctggtggcggtggatctggtggtggcggttctcaagtccaactggtgcaatccggcgccgaggtcaagaagccaggggcctcagtcaaagtgtcctgtaaagctagcggctatattttttctaattattggattcaatgggtgcgtcaggcccccgggcagggcctggaatggatgggtgagatcttaccgggctctggtagcaccgaatataccgaaaattttaaagaccgtgttactatgacgcgtgacacttcgactagtacagtatacatggagctctccagcctgcgatcggaggacacggccgtctattattgcgcgcgttatttttttggttctagcccgaattggtattttgatgtttggggtcaaggaaccctggtcactgtctcgagctga

SEQ ID NO: 22 - protein, 246 aa, Artificial Sequence
  source: /note=“Description of Artificial Sequence Synthetic polypeptide”
DIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTGGGGSGGGGSGGGGSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 23 - protein, 15 aa, Artificial Sequence
  source: /note=“Description of Artificial Sequence Synthetic peptide”
GGGGSGGGGSGGGGS

SEQ ID NO: 24 - protein, 1676 aa, Artificial Sequence
  source: /note=“Description of Artificial Sequence Synthetic polypeptide”
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVILNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGIGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 25 - protein, 448 aa, Artificial Sequence; identical to SEQ ID NO: 10
  source: /note=“Description of Artificial Sequence Synthetic polypeptide”

SEQ ID NO: 26 - protein, 214 aa, Artificial Sequence [sequence omitted]

SEQ ID NO: 27 - protein, 448 aa, Artificial Sequence; identical to SEQ ID NO: 14
  source: /note=“Description of Artificial Sequence Synthetic polypeptide”

(19) United States
(12) Patent Application Publication
(10) Pub. No.: US 2018/0142010 A1
(43) Pub. Date: May 24, 2018

(54) METHOD FOR TREATING A PATIENT IN COMPLIANCE WITH VACCINATION WITH ECULIZUMAB OR AN ECULIZUMAB VARIANT

(71) Applicant: Alexion Pharmaceuticals, Inc., New Haven, CT (US)

(72) Inventors: Leonard BELL, Woodbridge, CT (US); Camille BEDROSIAN, Woodbridge, CT (US)

(21) Appl. No.: 15/580,760

(22) PCT Filed: Jun. 22, 2016
(86) PCT No.: PCT/US2016/038756

§ 371 Date: Dec. 8, 2017

Related U.S. Application Data
(60) Provisional application No. 62/208,015, filed on Aug. 21, 2015.
(60) Provisional application No. 62/185,139, filed on Jun. 26, 2015.

(51) Int. Cl. C07K 16/18; A61K 39/095; A61K 39/395; A61P 27/02; A61P 37/06; A61P 19/02; A61P 11/00; A61P 9/00; A61P 7/00; A61P 7/02; A61P 13/12; A61P 15/06; A61P 17/00; A61P 25/28; A61P 21/04; A61P 29/00; A61P 5/14; A61P 3/10; A61P 17/06; A61P 25/02; A61P 31/04; A61P 31/12

(52) U.S. Cl. CPC C07K 16/18; A61K 2039/545; A61K 39/3955; A61P 27/02; A61P 37/06; A61P 19/02; A61P 11/00; A61P 9/00; A61P 7/00; A61P 7/02; A61P 13/12; A61P 15/06; A61P 17/00; A61P 25/28; A61P 21/04; A61P 29/00; A61P 5/14; A61P 3/10; A61P 17/06; A61P 25/02; A61P 31/04; A61P 31/12; C07K 2317/24; A61K 39/095

(57) ABSTRACT

The present disclosure relates to, inter alia, a method of treating a patient in need of treatment with a C5 inhibitor or a method for inhibiting formation of terminal complement in a patient, comprising administering an effective amount of a C5 inhibitor, such as eculizumab or an eculizumab variant, to a patient who is or will be in compliance with vaccination with a Neisseria meningococcal Type B specific vaccine.

### INCORPORATION OF SEQUENCE LISTING

The instant application contains a Sequence Listing, which has been submitted electronically in ASCII format and is hereby incorporated by reference in its entirety. Said ASCII copy, created on Jun. 22, 2016, is named AXJ-220PC_SL.txt and is 64,249 bytes in size.

### TECHNICAL FIELD

This application relates to the fields of immunology and infectious disease.

### BACKGROUND

Eculizumab is a humanized anti-human C5 monoclonal antibody (Alexion Pharmaceuticals, Inc.), with a human IgG2/IgG4 hybrid constant region, so as to reduce the potential to elicit proinflammatory responses. Eculizumab has the trade name Soliris(r) and is currently approved for treating paroxysmal nocturnal hemoglobinuria (“PNH”) and atypical hemolytic uremic syndrome (“aHUS”). Paroxysmal nocturnal hemoglobinuria is a form of hemolytic anemia, intravascular hemolysis being a prominent feature. AHUS involves chronic uncontrolled complement activation, resulting in, inter alia, inhibition of thrombotic microangiopathy, the formation of blood clots in small blood vessels throughout the body, and acute renal failure. Eculizumab specifically binds to human C5 protein and blocks the formation of the generation of the potent proinflammatory protein C5a. Eculizumab further blocks the formation of the terminal complement complex. Eculizumab treatment reduces intravascular hemolysis in patients with PNH and decreases complement levels in aHUS. See, e.g., Hillmen et al., N Engl J Med 2004; 350:552-9; Rother et al., Nature Biotechnology 2007; 25 (11): 1256-1264, Hillmen et al., N Engl J Med 2006, 355; 12, 1233-1243; Zuber et al., Nature Reviews Nephrology 8, 643-657 (2012)|doi:10.1038/nrneph.2012.214; U.S. Patent Publication Number 2012/0237515, and U.S. Pat. No. 6,355,245. Eculizumab has also been shown in a recent clinical trial to be effective for patients with Shiga-toxin-producing E. coli hemolytic uremic syndrome (“STEC-HUS”). See Alexion press release, “New Clinical Trial Data Show Substantial Improvement with Eculizumab (Soliris®) in Patients with STEC-HUS,” Saturday, Nov. 3, 2012. PNH, aHUS, and STEC-HUS are all diseases relating to inappropriate complement activation. See, e.g., Noris et al., Nat Rev Nephrol. 2012 November; 8 (11):622-33.doi:10.1038/nrneph.2012.195. Epub 2012 Sep. 18; Hillmen et al., N Engl J Med 2004; 350:6, 552-9; Mother et al., Nature Biotechnology 2007; 25 (11): 1256-1264; Hillmen et al., N Engl J Med 2006, 355; 12, 1233-1243; Zuber et al., Nature Reviews Nephrology 8, 643-657 (2012)|doi:10.1038/nrneph.2012.214.

Patients being treated by eculizumab are at greater risk than the general population of being infected by Neisseria meningitidis. Therefore, it is recommended that such patients comply with the most current Advisory Committee on Immunization Practices (ACIP) recommendations for meningococcal vaccination in patients with complement deficiencies. That advisory, however, does not currently include vaccinating against Neisseria meningitidis serogroup B. Moreover, even patients vaccinated with a meningococcal vaccine can still contract a meningococcal infection. In PNH clinical studies, two patients experienced meningococcal sepsis, even though both patients had previously received a meningococcal vaccine. In clinical studies among patients without PNH, meningococcal meningitis occurred in one unvaccinated patient. Meningococcal sepsis occurred in one previously vaccinated patient enrolled in the retrospective aHUS study during the post-study follow-up period.

### SUMMARY

This disclosure provides a solution to these issues by providing a method of treating a patient, such as a human patient, in need of treatment with a C5 inhibitor, such as eculizumab or an eculizumab variant. The method comprises administering an effective amount of a C5 inhibitor, such as eculizumab or an eculizumab variant, to a patient, wherein the patient is one: who has been vaccinated with a Neisseria meningococcal type B specific vaccine before the patient's treatment with eculizumab or an eculizumab variant; or who is vaccinated with a Neisseria meningococcal type B specific vaccine concurrently with the patient's first administration with eculizumab or an eculizumab variant; or who has been administered eculizumab or an eculizumab variant before being vaccinated with a Neisseria meningococcal type B specific vaccine and the patient is vaccinated with a Neisseria meningococcal type B specific vaccine immediately upon discovery that the patient has not been vaccinated with a Neisseria meningococcal type B specific vaccine; or who has been administered eculizumab or an eculizumab variant before being vaccinated with a Neisseria meningococcal type B specific vaccine and that administration is interrupted until the patient is vaccinated with a Neisseria meningococcal type B specific vaccine.

In another aspect, a method is provided for inhibiting formation of terminal complement in a patient, such as a human patient. The method comprises administering a C5 inhibitor, such as eculizumab or an eculizumab variant, to the patient in an amount effective to inhibit terminal complement in the patient, wherein the patient is one:
who has been vaccinated with a Neisseria meningococcal type B specific vaccine before the patient's treatment with eculizumab or an eculizumab variant; or
who is vaccinated with a Neisseria meningococcal type B specific vaccine concurrently with the patient's first administration with eculizumab or an eculizumab variant; or
who has been administered eculizumab or an eculizumab variant before being vaccinated with a Neisseria meningococcal type B specific vaccine and the patient is vaccinated with a Neisseria meningococcal type B specific vaccine immediately upon discovery that the patient has not been vaccinated with a Neisseria meningococcal type B specific vaccine; or
who has been administered eculizumab or an eculizumab variant before being vaccinated with a Neisseria meningococcal type B specific vaccine and that administration is interrupted until the patient is vaccinated with a Neisseria meningococcal type B specific vaccine.

In yet another aspect, a method is provided of vaccinating a patient being treated with a C5 inhibitor, such as eculizumab or an eculizumab variant. The method comprises administering a Neisseria meningococcal type B specific vaccine 14±3 days prior to the administration of the C5 inhibitor, such as eculizumab or an eculizumab variant, or after that period of time but before about 14 days after the first administration of the C5 inhibitor.

Numerous other aspects are provided in accordance with these and other aspects of the disclosure. Other features and aspects of the present disclosure will become more fully apparent from the detailed description, and the appended claims.

In one embodiment, the C5 inhibitor is an anti-C5 antibody. An exemplary anti-C5 antibody is eculizumab (Soliris®) comprising the heavy and light chains having the sequences shown in SEQ ID NOs:10 and 11, respectively, or antigen binding fragments and variants thereof. In other embodiments, the antibody comprises the heavy and light chain complementarity determining regions (CDRs) or variable regions (VRs) of antibody BNJ441. Accordingly, in one embodiment, the antibody comprises the CDR1, CDR2, and CDR3 domains of the heavy chain variable (VH) region of antibody BNJ441 having the sequence shown in SEQ ID NO:7, and the CDR1, CDR2 and CDR3 domains of the light chain variable (VL) region of antibody BNJ441 having the sequence shown in SEQ ID NO:8. In another embodiment, the antibody comprises CDR1, CDR2 and CDR3 heavy chain sequences as set forth in SEQ ID NOs:1, 2, and 3, respectively, and CDR1, CDR2 and CDR3 light chain sequences as set forth in SEQ ID NOs:4, 5, and 6, respectively. In another embodiment, the antibody comprises VH and VL regions having the amino acid sequences set forth in SEQ ID NO:7 and SEQ ID NO:8, respectively.

Another exemplary anti-C5 antibody is antibody BNJ441 (also known as ALXN1210) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of antibody BNJ441. Accordingly, in one embodiment, the antibody comprises the CDR1, CDR2, and CDR3 domains of the heavy chain variable (VH) region of antibody BNJ441 having the sequence shown in SEQ ID NO:12, and the CDR1, CDR2 and CDR3 domains of the light chain variable (VL) region of antibody BNJ441 having the sequence shown in SEQ ID NO:8. In another embodiment, the antibody comprises CDR1, CDR2 and CDR3 heavy chain sequences as set forth in SEQ ID NOs:19, 18, and 3, respectively, and CDR1, CDR2 and CDRS light chain sequences as set forth in SEQ ID NQs:4, 5, and 6, respectively.

In another embodiment, the antibody comprises VH and VL regions having the amino acid sequences set forth in SEQ ID NO:12 and SEQ ID NO:8, respectively.

In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

### DETAILED DESCRIPTION

As used herein, the word “a” or “plurality” before a noun represents one or more of the particular noun. For example, the phrase “a mammalian cell” represents “one or more mammalian cells.”

The term “antibody” is known in the art. The term “antibody” is sometimes used interchangeably with the term “immunoglobulin.” Briefly, it can refer to a whole antibody comprising two light chain polypeptides and two heavy chain polypeptides. Whole antibodies include different antibody isotypes including IgM, IgG, IgA, IgD, and IgE antibodies. The term “antibody” includes, for example, a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody. The antibody can also be an engineered protein or antibody-like protein containing at least one immunoglobulin domain (e.g., a fusion protein). The engineered protein or antibody-like protein can also be a bi-specific antibody or a tri-specific antibody, or a dimer, trimer, or multimer antibody, or a diabody, a, DVD-Ig, a CODV-Ig, an Affibody®, or a Nanobody®.

The term “antibody fragment,” “antigen-binding fragment,” or similar terms are known in the art and can, for example, refer to a fragment of an antibody that retains the ability to bind to a target antigen (e.g., human C5) and inhibit the activity of the target antigen. Such fragments include, e.g., a single chain antibody, a single chain Fv fragment (scFv), an Fd fragment, a Fab fragment, a Fab′ fragment, or an F(ab′)2 fragment. A scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al. (2001) J Immunol Methods 248 (1):47-66; Hudson and Kortt (1999) J Immunol Methods 231 (1):177-189; Poljak (1994) Structure 2 (12):1121-1123; Rondon and Marasco (1997) Annual Review of Microbiology 51:257-283. An antigen-binding fragment can also include the variable region of a heavy chain polypeptide and the variable region of a light chain polypeptide. An antigen-binding fragment can thus comprise the CDRs of the light chain and heavy chain polypeptide of an antibody.

The term “antibody fragment” also can include, e.g., single domain antibodies such as camelized single domain antibodies. See, e.g., Muyldermans et al. (2001) Trends Biochem Sci 26:230-235; Nuttall et al. (2000) Curr Pharm Biotech 1:253-263; Reichmann et al. (1999) J Immunol Meth 231:25-38; PCT application publication nos. WO 94/04678 and WO 94/25591; and U.S. Pat. No. 6,005,079. The term “antibody fragment” also includes single domain antibodies comprising two V_{H} domains with modifications such that single domain antibodies are formed.

The term “subject” is used interchangeably with the term “patient.”

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As is well known, the complement system acts in conjunction with other immunological systems of the body to defend against intrusion of cellular and viral pathogens. There are at least 25 complement proteins. Complement components achieve their immune defensive functions by interacting in a series of intricate but precise enzymatic cleavage and membrane binding events. The resulting complement cascade leads to the production of products with opsonic, immunoregulatory, and lytic functions.

The complement cascade can progress via the classical pathway (“CP”), the lectin pathway, or the alternative pathway (“AP”). These pathways converge at the C3 convertase—the point where complement component C3 is cleaved by an active protease to yield C3a and C3b.

The AP C3 convertase is initiated by the spontaneous hydrolysis of complement component C3, which is abundant in the plasma in the blood. This process, also known as “tickover,” occurs through the spontaneous cleavage of a thioester bond in C3 to form C3i or C3(H₂O). This formation of C3(H₂O) allows for the binding of plasma protein Factor B, which in turn allows Factor D to cleave Factor B into Ba and Bb. The Bb fragment remains bound to C3 to form a complex containing C3(H₂O)Bb—the “fluid-phase” or “initiation” C3 convertase. Although only produced in small amounts, the fluid-phase C3 convertase can cleave multiple C3proteins into C3a and C3b and results in the generation of C3b and its subsequent covalent binding to a surface (e.g., a bacterial surface). Factor B bound to the surface-bound C3b is cleaved by Factor D to thus form the surface-bound AP C3 convertase complex containing C3b,Bb. See, e.g., Müller-Eberhard (1988) Ann Rev Biochem 57:321-347.

The AP C5 convertase—(C3b)₂, Bb—is formed upon addition of a second C3b monomer to the AP C3 convertase. See, e.g., Medicus et al. (1976) J Exp Med 144:1076-1093 and Fearon et al. (1975) J Exp Med 142:856-863. The role of the second C3b molecule is to bind C5 and present it for cleavage by Bb. See, e.g., Isenman et al. (1980) J Immunol 124:326-331. The AP C3 and C5 convertases are stabilized by the addition of the trimeric protein properdin as described in, e.g., Medicus et al. (1976), supra. However, properdin binding is not required to form a functioning alternative pathway C3 or C5 convertase. See, e.g., Schreiber et al. (1978) Proc Natl Acad Sci USA 75:3948-3952, and Sissons et al. (1980) Proc Natl Acad Sci USA 77: 559-562.

The CP C3 convertase is formed upon interaction of complement component C1, which is a complex of C1q, C1r, and C1s, with an antibody that is bound to a target antigen (e.g., a microbial antigen). The binding of the C1q portion of C1 to the antibody-antigen complex causes a conformational change in C1 that activates C1r. Active Or then cleaves the C1-associated C1s to thereby generate an active serine protease. Active C1s cleaves complement component C4 into C4b and C4a. Like C3b, the newly generated C4b fragment contains a highly reactive thiol that readily forms amide or ester bonds with suitable molecules on a target surface (e.g., a microbial ceil surface). C1s also cleaves complement component C2 into C2b and C2a. The complex formed by C4b and C2a is the CP C3 convertase, which is capable of processing C3 into C3a and C3b. The CP C5 convertase—C4b,C2a,C3b—is formed upon addition of a C3b monomer to the CP C3 convertase. See, e.g., Müller-Eberhard (1988), supra and Cooper et al. (1970) J Exp Med 132:775-793.

C3b also functions as an opsonin through its interaction with complement receptors present on the surfaces of antigen-presenting cells such as macrophages and dendritic cells. The opsonic function of C3b is generally considered to be one of the most important anti-infective functions of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection.

The AP and CP C5 convertases cleave C5. C5 can also be activated by means other than C5 convertase activity, such as limited trypsin digestion (see, e.g., Minta and Man (1997) J Immunol 119:1597-1602 and Wetsel and Kolb (1982) J Immunol 128:2209-2216). And acid treatment (Yamamoto and Gewurz (1978) J Immunol 120:2008 and Damerau et al. (1989) Molec Immunol 26:1133-1142) can also cleave C5 and produce active C5b.

Cleavage of C5 releases C5a, a potent anaphylatoxin and chemotactic factor, and leads to the formation of the lytic terminal complement complex, C5b-9. C5a and C5b-9 also have pleiotropic cell activating properties, by amplifying the release of downstream inflammatory factors, such as hydrolytic enzymes, reactive oxygen species, arachidonic acid metabolites and various cytokines.

C3a and C5a are anaphylatoxins. These activated complement components can trigger mast cell degranulation, which releases histamine from basophils and mast cells, and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation.

While a properly functioning complement system provides a robust defense against infecting microbes, inappropriate regulation or activation of complement has been implicated in the pathogenesis of a variety of disorders, including, e.g., rheumatoid arthritis (“RA”); lupus nephritis; asthma; ischemia-reperfusion injury; atypical hemolytic uremic syndrome (“aHUS”); dense deposit disease (“DDD”); paroxysmal nocturnal hemoglobinuria (“PNH”); macular degeneration (e.g., age-related macular degeneration (“AMD”)); hemolysis, elevated liver enzymes, and low platelets (“HELLP”) syndrome; thrombotic thrombocytopenic purpura (“TTP”); spontaneous fetal loss; Pauci-immune vasculitis; epidermolysis bullosa; recurrent fetal loss; multiple sclerosis (“MS”); traumatic brain injury; sepsis, viral hemorrhagic fever (such as Ebola hemorrhagic fever), and injury resulting from myocardial infarction, cardiopulmonary bypass and hemodialysis. See, e.g., Holers et al. (2008) Immunological Reviews 223:300-316. Inhibition of complement (e.g., inhibition of terminal complement formation, C5 cleavage, or complement activation) has been demonstrated to be effective in treating several complement-associated disorders both in animal models and in humans. See, e.g., Rother et al. (2007) Nature Biotechnology 25 (11):1256-1264; Wang et al. (1996) Proc Natl Acad Sci USA 93:8563-8568, Wang et al. (1995) Proc Natl Acad Sci USA 92:8955-8959; Rinder et al. (1995) J Clin Invest 96:1564-1572, Kroshus et al. (1995) Transplantation 60:1194-1202; Homeister et al. (1993) J Immunol 150:1055-1064; Weisman et al. (1990) Science 249:146-151; Amsterdam et al. (1995) Am J Physiol 268:H448-H457; and Rabinovici et al. (1992) J Immunol 149:1744 1750.

It is well known that complement deficient individuals are more susceptible to meningococcal infections; and thus it is recommended that such individuals be vaccinated against Neisseria meningitidis. See, e.g., Figueroa et al., Clinical Microbiology Reviews, July 1991, Vol. 4, No. 3, p. 359-395.

For Soliris® (i.e., eculizumab), meningococcal infections are the most important adverse reactions experienced by patients while on the drug. In PNH clinical studies, the use of Soliris® increases a patient's susceptibility to serious meningococcal infections (septicemia and/or meningitis). The risk groups or the most known risk factors include: 1) genetic deficiency or therapeutic inhibition of terminal complement (such as Soliris® therapy); 2) lack of commercially available vaccine against meningococcal serogroup B (now available), and 3) delay or absence of appropriate medical consultation at the appearance of first symptoms. The occurrence of meningococcal infection can be prevented in some cases by means of meningococcal vaccines. For example, patients without a history of meningococcal vaccination can be vaccinated at least 2 weeks prior to receiving the first dose of Soliris® or other complement inhibitor. If urgent Soliris® therapy is indicated in an unvaccinated patient, the meningococcal vaccine should be administered as soon as possible. In patients who cannot receive meningococcal vaccine, including children below the age of two years, antibiotic prophylaxis could prevent meningococcal infection. However, meningococcal vaccination reduces, but does not eliminate, the risk of meningococcal infections. In addition, previously available meningococcal vaccines do not cover all serogroups, notably serogroup B infection. In clinical studies, 2 out of 196 PNH patients developed serious meningococcal infections while receiving treatment with Soliris®, both of whom had been vaccinated. In clinical studies among non-PNH patients, meningococcal meningitis occurred in one unvaccinated patient. In addition, a previously vaccinated patient with aHUS developed meningococcal sepsis during the post-study follow-up period.

Since anti-C5 antibodies or antigen-binding fragments (e.g., eculizumab and pexelizumab) block terminal complement activation, patients treated with these agents (e.g., eculizumab/Soliris®) may have increased susceptibility to infections in addition to meningococcal infections, especially with encapsulated bacteria. For example, children or adolescent patients may be at increased risk of developing serious infections due to Streptococcus pneumonia and Haemophilus influenza type B (1 lib). In clinical studies, a total of 11 out of 195 PNH patients experienced an infection-related serious adverse event (SAE) with eculizumab treatment, including Cellulitis (1 patient), Haemophilus infection (1 patient), other infection (1 patient), Meningococcal sepsis (2 patients), Necrotizing fasciitis (1 patient), respiratory tract infection (1 patient), urinary tract infection (1 patient), viral infection (2patients), and viral upper respiratory tract infection (1 patient). One out of 37 aHUS patients treated with eculizumab was found to have peritonitis. Correspondingly, vaccinations for the prevention of these infections should be administered prior to the treatment by terminal complement C5 inhibition.

In one aspect, a method is provided of treating a patient, such as a human patient, in need of treatment with a C5 inhibitor, such as eculizumab or an eculizumab variant. The method comprises administering an effective amount of a C5 inhibitor, such as eculizumab or an eculizumab variant, to a patient, wherein the patient is one: who has been vaccinated with a Neisseria meningococcal type B specific vaccine before the patient's treatment with a C5 inhibitor, such as eculizumab or an eculizumab variant; or who is vaccinated with a Neisseria meningococcal type B specific vaccine concurrently with the patient's first administration with a C5 inhibitor, such as eculizumab or an eculizumab variant; or who has been administered a C5inhibitor, such as eculizumab or an eculizumab variant, before being vaccinated with a Neisseria meningococcal type B specific vaccine and the patient is vaccinated with a Neisseria meningococcal type B specific vaccine immediately upon discovery that the patient has not been vaccinated with a Neisseria meningococcal type B specific vaccine; or who has been administered a C5 inhibitor, such as eculizumab or an eculizumab variant before being vaccinated with a Neisseria meningococcal type B specific vaccine and that administration is interrupted until the patient is vaccinated with a Neisseria meningococcal type B specific vaccine.

In another aspect, a method is provided for inhibiting formation of terminal complement in a patient, such as a human patient. The method comprises administering a C5 inhibitor, such as eculizumab or an eculizumab variant, to the patient in an amount effective to inhibit terminal complement in the patient; wherein the patient is one: who has been vaccinated with a Neisseria meningococcal type B specific vaccine before the patient's treatment with a C5 inhibitor, such as eculizumab or an eculizumab variant; or who is vaccinated with a Neisseria meningococcal type B specific vaccine concurrently with the patient's first administration with a C5 inhibitor, such as eculizumab or an eculizumab variant; or who has been administered a C5 inhibitor, such as eculizumab or an eculizumab variant, before being vaccinated with a Neisseria meningococcal type B specific vaccine and the patient is vaccinated with a Neisseria meningococcal type B specific vaccine immediately upon discovery that the patient has not been vaccinated with a Neisseria meningococcal type B specific vaccine; or
who has been administered a C5 inhibitor, such as eculizumab or an eculizumab variant before being vaccinated with a Neisseria meningococcal type B specific vaccine and that administration is interrupted until the patient is vaccinated with a Neisseria meningococcal type B specific vaccine.

In yet another aspect, a method is provided of vaccinating a patient, such as a human patient, being treated with a C5 inhibitor, such as eculizumab or an eculizumab variant. The method comprises administering a Neisseria meningococcal type B specific vaccine 14±3 days prior to the administration of the C5 inhibitor, such as eculizumab or an eculizumab variant, or after that period of time but about 14 days after the first administration of the C5 inhibitor.

The patients are monitored for meningitis by methods known in the art.

In certain embodiments, the patient has been diagnosed with paroxysmal nocturnal hemoglobinuria (“PNH”), atypical hemolytic uremic syndrome (“aHUS”), or Shiga-toxin-producing E. coli hemolytic uremic syndrome (“STEC-HUS”).

In certain embodiments, the patient suffers from a complement-associated disorder. The complement-associated disorder can be any complement-associated disorder. The complement-associated disorder includes, for example, age-related macular degeneration, graft rejection, bone marrow rejection, kidney graft rejection, skin graft rejection, heart graft rejection, lung graft rejection, liver graft rejection, rheumatoid arthritis, a pulmonary condition, ischemia-reperfusion injury, atypical hemolytic uremic syndrome, thrombotic thrombocytopenic purpura, paroxysmal nocturnal hemoglobinuria, dense deposit disease, age-related macular degeneration, spontaneous fetal loss, Pauci-immune vasculitis, epidermolysis bullosa, recurrent fetal loss, multiple sclerosis, traumatic brain injury, myasthenia gravis, cold agglutinin disease, dermatomyositis, Degos' disease, Graves' disease, Hashimoto's thyroiditis, type I diabetes, psoriasis, pemphigus, autoimmune hemolytic anemia, idiopathic thrombocytopenic purpura, Goodpasture syndrome, multifocal motor neuropathy, neuromyelitis optica, antiphospholipid syndrome, sepsis, viral hemorrhagic fever (such as Ebola hemorrhagic fever), and catastrophic antiphospholipid syndrome.

A Neisseria meningococcal type B specific vaccine can be any meningococcal vaccine to Neisseria meningitidis serogroup B. In certain embodiments, the Neisseria meningococcal type B specific vaccine is multicomponent meningococcal serogroup B vaccine (4CMenB or BEXSERO®) or meningococcal group B vaccine (Neisseria meningitidis serogroup B recombinant 1p2086 a05 protein variant antigen and Neisseria meningitidis serogroup B recombinant 1p2086 b01 protein variant antigen, or Trumenba® ) (see U.S. Pat. No. 8,563,006).

In certain embodiments, the recommended indication and usage, dosage and administration, dosage forms and strength, and use in specific patient population of either BEXSERO® or Trumenba® should be followed. However, a healthcare professional may adjust the recommended indication and usage, dosage and administration, dosage forms and strength, and use in specific patient population of either BEXSERO® or Trumenba® as needed.

In certain embodiments, the patient has been, or is or will be vaccinated concurrently or during the patient's treatment with a complement inhibitor, such as eculizumab or an eculizumab variant, with one or more additional meningococcal vaccine, including MPSV4, MenACWY, MenACWY-D, MenACWY-CRM, or HibMenCY-TT.

In certain embodiments, the meningococcal vaccine to Neisseria Meningitidis serogroup B is administered to the patient prior to administering the C5 inhibitor, such as eculizumab or the eculizumab variant, to the patient.

In certain embodiments, “vaccination,” “administering a vaccine,” or the like, as used herein, refers to having fully complied with the dosage and frequency of administration as recommended by the manufacturer of the vaccine.

In certain embodiments, any administration of a meningococcal vaccine to the patient is performed prior to administering the C5 inhibitor, such as eculizumab or the eculizumab variant, to the patient.

In certain embodiments, the patient is one who has been vaccinated with a Neisseria meningococcal type B specific vaccine before the patient's treatment with a C5 inhibitor, such as eculizumab or an eculizumab variant.

In certain embodiments, the patient is one who is vaccinated with a Neisseria meningococcal type B specific vaccine concurrently with the patient's first administration with a C5 inhibitor, such as eculizumab or an eculizumab variant.

In certain embodiments, the patient is one who has been administered a C5 inhibitor, such as eculizumab or an eculizumab variant, before being vaccinated with a Neisseria meningococcal type B specific vaccine and the patient is vaccinated with a Neisseria meningococcal type B specific vaccine immediately upon discovery that the patient has not been vaccinated with a Neisseria meningococcal type B specific vaccine.

In certain embodiments, the patient is one who has been administered a C5 inhibitor, such as eculizumab or an eculizumab variant, before being vaccinated with a Neisseria meningococcal type B specific vaccine and that administration is interrupted until the patient is vaccinated with a Neisseria meningococcal type B specific vaccine.

In certain embodiments, a patient can be vaccinated with one of the Neisseria meningococcal type B specific vaccine at one time and with another one of the Neisseria meningococcal type B specific vaccine at another time. For example, a patient can be vaccinated with BEXSERO® before the patient's treatment with eculizumab or an eculizumab variant and then be vaccinated with Trumenba® concurrently with the patient's first administration with eculizumab or an eculizumab variant.

The methods disclosed herein can be practiced by administering a complement C5 inhibitor other than eculizumab or an eculizumab variant. In certain embodiments, the C5 inhibitor inhibits human C5. A C5 inhibitor for use in a method of this invention can be any C5 inhibitor. In certain embodiments, the C5 inhibitor for use in methods disclosed herein is eculizumab, an antigen-binding fragment thereof, a polypeptide comprising the antigen-binding fragment of eculizumab, a fusion protein comprising the antigen binding fragment of eculizumab, or a single chain anti body version of eculizumab, or a small-molecule C5 inhibitor. In certain embodiments, the C5 inhibitor inhibits human C5.

In some embodiments, the C5 inhibitor is a small-molecule chemical compound. One example of a small molecule chemical compound that is a C5 inhibitor is Aurin tricarboxylic acid. In other embodiments, the C5 inhibitor is a polypeptide.

The C5 inhibitor is one that binds to a complement C5 protein and is also capable of inhibiting the generation of C5a. A C5-binding inhibitor can also be capable of inhibiting, e.g., the cleavage of C5 to fragments C5a and C5b, and can thus prevent the formation of terminal complement complex. In some embodiments, the C5 inhibitor is a polypeptide inhibitor. In one embodiment, the C5 inhibitor is an anti-C5 antibody. An exemplary anti-C5 antibody is eculizumab (Soliris®; Alexion Pharmaceuticals, Inc., Cheshire, Conn.), or an antibody that binds to the same epitope on C5 as or competes for binding to C5 with eculizumab (See, e.g., Kaplan (2002) Curr Opin Investig Drugs 3 (7):1017-23; Hill (2005) Clin Adv Hematol Oncol 3 (11):849-50; and Rother et al. (2007) Nature Biotechnology 25 (11):1256-1488). Soliris®, is a formulation of eculizumab which is a recombinant humanized monoclonal IgG2/4K. antibody produced by murine myeloma cell culture and purified by standard bioprocess technology. Eculizumab contains human constant regions from human IgG2 sequences and human IgG4 sequences and murine complementarity-determining regions grafted onto the human framework light- and heavy-chain variable regions. Eculizumab is composed of two 448 amino acid heavy chains and two 214 amino acid light chains and has a molecular weight of approximately 148 kDa. Eculizumab comprises the heavy and light chain amino acid sequences set forth in SEQ ID NOs: 10 and 11, respectively; heavy and light chain variable region amino acid sequences set forth in SEQ ID NOs: 7 and 8, respectively; and heavy chain variable region CDR1-3 and light chain variable region CDR1-3 sequences set forth in SEQ ID NOs: 1, 2, and 3 and 4, 5, and 6, respectively.

Eculizumab is currently approved for treating paroxysmal nocturnal hemoglobinuria (“PNH”) and atypical hemolytic uremic syndrome (“aHUS”). Paroxysmal nocturnal hemoglobinuria is a form of hemolytic anemia, intravascular hemolysis being a prominent feature due to the absence of the complement regulatory protein CD59 and CD55. CD59, for example, functions to block the formation of the terminal complement complex. AHUS involves chronic uncontrolled complement activation, resulting in, inter alia, inhibition of thrombolitic microangiopathy, the formation of blood clots in small blood vessels throughout the body, and acute renal failure. Eculizumab specifically binds to human C5 protein and blocks the formation of the generation of the potent proinflammatory protein C5a. Eculizumab further blocks the formation of the terminal complement complex. Eculizumab treatment reduces intravascular hemolysis in patients with PNH and decreases complement levels in aHUS. See, e.g., Hillmen et al., N Engl J Med 2004; 350:552-9; Rother et al., Nature Biotechnology 2007; 25 (11): 1256-1264; Hillmen et al., N Engl J Med 2006, 355; 12, 1233-1243; Zuber et al, Nature Reviews Nephrology 8, 643-657 (2012)|doi:10.1038/nrneph.2012.214; U.S. Patent Publication Number 2012/0237515, and U.S. Pat. No. 6,355,245. Eculizumab has also been shown in a recent clinical trial to be effective for patients with Shiga-toxin-producing E. coli hemolytic uremic syndrome (“STEC-HUS”). See Alexion press release, “New Clinical Trial Data Show Substantial Improvement with Eculizumab (Soliris®) in Patients with STEC-HUS,” Saturday, Nov. 3, 2012. STEC-HUS is characterized by systemic complement-mediated thrombotic microangiopathy and acute vital organ damage. Eculizumab administration to these patients resulted in rapid and sustained improvement in thrombotic microangiopathy and improvements in systemic organ complications. PNH, aHUS, and STEC-HUS are all diseases relating to inappropriate complement activation. See, e.g., Noris et al., Nat Rev Nephrol. 2012 November; 8 (11):622-33.doi: 10.1038/nrneph.2012.195. Epub 2012 Sep. 18; Hillmen et al., N Engl J Med, 2004, 350:6, 552-9; Rother et al., Nature Biotechnology 2007; 25 (11): 1256-1264; Hillmen et al., N Engl J Med 2006, 355; 12, 1233-1243; Zuber et al., Nature Reviews Nephrology 8, 643-657 (2012)|doi:10.1038/nrneph.2012.214.

Another exemplary anti-C5 antibody is antibody BNJ441 comprising heavy and light having the sequences shown in SEQ ID NQs:14 and 11, respectively, or antigen binding fragments and variants thereof. BNJ441 (also known as ALXN1210) is described in PCT/US2015/019225 and U.S. Pat. No. 9,079,949, the teachings or which are hereby incorporated by reference. BNJ441 is a humanized monoclonal antibody that is structurally related to eculizumab (Soliris®). BNJ441 selectively binds to human complement protein C5, inhibiting its cleavage to C5a and C5b during complement activation. This inhibition prevents the release of the proinflammatory mediator C5a and the formation of the cytolytic pore-forming membrane attack complex C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of BNJ441. Accordingly, in one embodiment, the antibody comprises the CDR1, CDR2, and CDR3 domains of the VH region of BNJ441 having the sequence set forth in SEQ ID NO:12, and the CDR1, CDR2 and CDR3 domains of the VL region of BNJ441 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VI. regions having the amino acid sequences set forth in SEQ ID NO:12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in PCT/US2015/019225 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.], In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33 (17/18):1389-1401] exemplifies the identification of CDR boundaries according to Kabat and Chothia definitions.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVY
YCARYFFGSSPNWYFDVWGQGTLVTVSS.

In some embodiments, an anti-C5 antibody described herein comprises a light chain variable region comprising the following amino acid sequence:

(SEQ ID NO: 8)
DIQMTQSPSSLSASVGDRVTITCGASENIYGALNWYQQKPGKAPKLL
IYGATNLADGVPSRFSGSGSGTDFTLTISSLQPEDFATYYCQNVLNT
PLTFGQGTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. For example, the Fc constant region can comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., PCT/US2015/019225 and U.S. Pat. No. 9,079, 949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution described by Dall' Acqua et al. (2006) J Biol Chem 281:23514-23524; (2) the M428L or T250Q/M428L substitutions described in Hinton et al. (2004) J Biol Chem 279:6213-6216 and Hinton et al. (2006) J Immunol 176:346-356; and (3) the N434A or T307/E380A/N434A substitutions described in Petkova et al. (2006) Int. Immunol 18 (12):1759-69. The additional substitution pairings: P257I/Q311I, P257I/N434H, and D376V/N434H are described in, e.g., Datta-Mannan et al. (2007) J Biol Chem 282 (3):1709-1717, the disclosure of which is incorporated herein by reference in its entirety.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three, or two) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I, and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in BNJ441 and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, or 436 (EU numbering) relative to the native human Fc constant region. In some embodiments, the substitution is selected from the group consisting of: methionine for glycine at position 237; alanine for proline at position 238; lysine for serine at position 239, isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252, threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303, alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332, leucine for lysine at position 334; histidine for lysine at position 360; alanine for aspartic acid at position 376; alanine for glutamic acid at position 380; alanine for glutamic acid at position 382, alanine for asparagine or serine at position 384; aspartic acid or histidine for glycine at position 385; proline for glutamine at position 386; glutamic acid for proline at position 387; alanine or serine for asparagine at position 389; alanine for serine at position 424, alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable an anti-C5 antibodies for use in the methods described herein, in some embodiments, comprise a heavy chain polypeptide comprising the amino acid sequence depicted in SEQ ID NO:14 and/or a light chain polypeptide comprising the amino acid sequence depicted in SEQ ID NO:11. Alternatively, the anti-C5 antibodies for use in the methods described herein, in some embodiments, comprise a heavy chain polypeptide comprising the amino acid sequence depicted in SEQ ID NO:20 and/or a light chain polypeptide comprising the amino acid sequence depicted in SEQ ID NO:11.

Anti-C5 antibodies, or antigen-binding fragments thereof described herein, used in the methods described herein can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (see, Kohler & Mil stein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989). The anti-C5 antibodies, or antigen binding fragments thereof, can be administered to a patient by any suitable means. In one embodiment, the antibodies are formulated for intravenous administration.

In yet further other embodiments, the C5 inhibitor is a single chain version of eculizumab, including pexelizumab (SEQ ID NO:21)—a specific single chain version of the whole antibody eculizumab. See, e.g., Whiss (2002) Curr Opin Investig Drugs 3 (6):870-7; Patel et al. (2005) Drugs Today (Barc) 41 (3):165-70, Thomas et al. (1996) Mol Immunol 33 (17-18):1389-401; and U.S. Pat. No. 6,355,245. In yet other embodiments, the inhibitor for use in methods of this invention is a single chain variant of pexelizumab, with the arginine (R) at position 38 (according to Kabat numbering and the amino acid sequence number set forth in SEQ ID NO:22) of the light chain of the pexelizumab antibody amino acid sequence changed to a glutamine (Q). The single chain antibody having the amino acid sequence depicted in SEQ ID NO:22 is a variant of the single chain antibody pexelizumab (SEQ ID NG:21), in which the arginine (R) at position 38 has been substituted with a glutamine (Q). An exemplary linker amino acid sequence present in a variant pexelizumab antibody is shown in SEQ ID NG:23.

In certain embodiments, the anti-C5 antibody for use in methods disclosed herein is a variant derived from eculizumab, having one or more improved properties (e.g., improved pharmacokinetic properties) relative to eculizumab. The variant eculizumab antibody (also referred to herein as an eculizumab variant, a variant eculizumab, or the like) or C5-binding fragment thereof is one that: (a) binds to complement component C5; (b) inhibits the generation of C5a; and can further inhibit the cleavage of C5 into fragments C5a and C5b. The variant eculizumab antibody can have a serum half-life in a human that is greater than, or at least, 10 (e.g., greater than, or at least, 11, 12, 13, 14, 15, 16, 17, 18, 19, 20, 21, 22, 23, 24, 25, 26, 27, 28, 29, 30, 31, 32, 33 or 34) days. Such variant eculizumab antibodies are described in U.S. Pat. No. 9,079,949.

In certain embodiments, the eculizumab variant antibody is an antibody defined by the sequences depicted in SEQ ID NO:27 (heavy chain) and SEQ ID NQ:26 (light chain), or an antigen-binding fragment thereof. This antibody binds to human C5 and inhibits the formation of C5a, as well as the cleavage of C5 to fragments C5a and C5b, and thus preventing the formation of terminal complement complex.

In some embodiments, a C5-binding polypeptide for use in the methods disclosed herein is not a whole antibody. In some embodiments, a C5-binding polypeptide is a single chain antibody. In some embodiments, a C5-binding polypeptide for use in the methods disclosed herein is a bispecific antibody. In some embodiments, a C5-binding polypeptide for use in the methods disclosed herein is a humanized monoclonal antibody, a chimeric monoclonal antibody, or a human monoclonal antibody, or an antigen binding fragment of any of them.

Methods of making a polypeptide C5 inhibitor, including antibodies, are known in the art.

The C5-binding polypeptide for use in methods disclosed herein can comprise, or can consist of, the amino acid sequence depicted in SEQ ID NO:21, SEQ ID NO:22, SEQ ID NO:24, SEQ ID NO:25, SEQ ID NO:26, or SEQ ID NO: 27, or an antigen binding fragment of any of the above. The polypeptide can comprise one or more of the amino acid sequence depicted in SEQ ID NOs: 1-8.

In yet other embodiments, the C5 inhibitor is LFG316 (Novartis, Basel, Switzerland, and MorphoSys, Planegg, Germany) or another antibody defined by the sequences of Table 1 in U.S. Pat. No. 8,241,628 and U.S. Pat. No. 8,883,158, ARC 1905 (Ophthotech, Princeton, N.J. and New York, N.Y.), which is an anti-C5 pegylated RNA aptamer (see, e.g., Keefe et al., Nature Reviews Drug Discovery 9, 537-550 (July 2010) doi:10.1038/nrd3141), Mubodina® (Adienne Pharma & Biotech, Bergamo, Italy) (see, e.g., U.S. Pat. No. 7,999,081), rEV576 (coversin) (Volution Immuno-pharmaceuticals, Geneva, Switzerland) (e.g., Penabad et al., Lupus, 2014 October; 23 (12):1324-6. doi: 10.1177/0961203314546022.), ARC 1005 (Novo Nordisk, Bagsvaerd, Denmark), SOMAmers (SomaLogic, Boulder, Colo.), SOB 1002 (Swedish Orphan Biovitrum, Stockholm, Sweden), RA101348 (Ra Pharmaceuticals, Cambridge, Mass.), Aurin Tricarboxylic Acid (“ATA”), and anti-C5-siRNA (Alnylam Pharmaceuticals, Cambridge, Mass.), and Ornithodoros moubata C inhibitor (‘OmCI”).

Suitable methods for measuring inhibition of C5 cleavage are known in the art. For example, the concentration and/or physiologic activity of C5a and/or C5b in a body fluid can be measured by methods well known in the art. Methods for measuring C5a concentration or activity include, e.g., chemotaxis assays, RIAs, or ELISAs (see, e.g., Ward and Zvaifler (1971) J Clin Invest 50 (3):606-16 and Wurzner et al. (1991) Complement Inflamm 8:328-340). For C5b, hemolytic assays or assays for soluble C5b-9 known in the art can be used. Other assays known in the art can also be used.

For those C5 inhibitors that also inhibit TCC formation, inhibition of complement component C5 can also reduce the cell lysing ability of complement in a subject's body fluids. Such reductions of the cell-lysing ability of complement present can be measured by methods well known in the art such as, for example, by a conventional hemolytic assay such as the hemolysis assay described by Kabat and Mayer (eds), “Experimental Immunochemistry, 2^{nd} Edition,” 135-240, Springfield, Ill., C C Thomas (1961), pages 135-139, or a conventional variation of that assay such as the chicken erythrocyte hemolysis method as described in, e.g., Hillmen et al. (2004) N Engl J Med 350 (6):552.

In some embodiments, the C5-binding polypeptides for use in methods disclosed herein are variant antibodies of an anti-C5 antibody (such as eculizumab) that still bind to the antigen, including deletion variants, insertion variants, and/or substitution variants. See, e.g., the polypeptides depicted in SEQ ID NO:21, SEQ ID NO:22, or SEQ ID NO:27. Methods of making such variants, by, for example, recombinant DNA technology, are well known in the art.

In some embodiments, a C5-binding polypeptide for use in a method disclosed herein is a fusion protein. The fusion protein can be constructed recombinantly such that the fusion protein is expressed from a nucleic acid that encodes the fusion protein. The fusion protein can comprise one or more C5-binding polypeptide segments (e.g., C5-binding segments depicted in SEQ ID NO:21, SEQ ID NO:22, SEQ ID NO:24, SEQ ID NO:25 and/or SEQ ID NO:26, and/or SEQ ID NO:27, or any one or more of SEQ ID NOs: 1-8) and one or more segments that are heterologous to the C5-binding segment(s). The heterologous sequence can be any suitable sequence, such as, for example, an antigenic tag (e.g., FLAG, polyhistidine, hemagglutinin (“HA”), glutathione-S-transferase (“GST”), or maltose-binding protein (“MBP”)). Heterologous sequences can also be proteins useful as diagnostic or detectable markers, for example, luciferase, green fluorescent protein (“GFP”), or chloramphenicol acetyl transferase (“CAT”). In some embodiments, the heterologous sequence can be a targeting moiety that targets the C5-binding segment to a cell, tissue, or microenvironment of interest. In some embodiments, the targeting moiety is a soluble form of a human complement receptor (e.g., human complement receptor 2) or an antibody (e.g., a single chain antibody) that binds to C3b or C3d. In some embodiments, the targeting moiety is an antibody that binds to a tissue-specific antigen, such as a kidney-specific antigen. Methods of constructing such fusion proteins, such as by recombinant DNA technology, are well known in the art.

In some embodiments, the C5-binding polypeptides are fused to a targeting moiety. For example, a construct can contain a C5-bitiding polypeptide and a targeting moiety that targets the polypeptide to a site of complement activation. Such targeting moieties can include, e.g., soluble form of complement receptor 1 (CR1), a soluble form of complement receptor 2 (CR2), or an antibody (or antigen-binding fragment thereof) that binds to C3b and/or C3d.

Methods for generating fusion proteins (e.g., fusion proteins containing a C5-binding polypeptide and a soluble form of human CR1 or human CR2), including recombinant DNA technology, are known in the art and described in, e.g., U.S. Pat. No. 6,897,290; U.S. patent application publication no. 2005265995, and Song et al. (2003) J Clin Invest 11 (12):1875-1885.

In certain embodiments, the C5 inhibitor is a bispecific antibody. Methods for producing abispecific antibody (e.g., a bispecific antibody comprising an anti-C5 antibody and an antibody that binds to C3b and/or C3d) are also known in the art. A bispecific antibody comprising a C5-binding antibody and any other antibody is contemplated.

Methods of making, identifying, purifying, modifying, using etc, a C5 inhibitor for use in methods disclosed herein are well known in the art. For instance, C5 inhibitors that are small molecule chemical compounds can be produced by methods known in the art. The C5-binding inhibitors, including polypeptides and antibodies, used in the methods of this invention can be produced using a variety of techniques known in the art of molecular biology and protein chemistry.

Compositions containing a C5 inhibitor, such as a C5-binding polypeptide, can be formulated as a pharmaceutical composition. Any suitable pharmaceutical compositions and formulations, as well as suitable methods for formulating and suitable routes and suitable sites of administration, are within the scope of this invention, and are known in the art. Also, any suitable dosage(s) and frequency of administration are contemplated.

The pharmaceutical compositions can include a pharmaceutically acceptable carrier. A “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see e.g., Berge et al. (1977) J Pharm Sci 66:1-19).

In certain embodiments, the protein compositions can be stabilized and formulated as a solution, microemulsion, dispersion, liposome, lyophilized (freeze-dried) powder, or other ordered structure suitable for stable storage at high concentration. Sterile injectable solutions can be prepared by incorporating a C5-binding polypeptide, for use in the methods of this invention, in the required amount in an appropriate solvent with one or a combination of ingredients enumerated above, as required, followed by filtered sterilization. The C5 inhibitor, including a C5-binding polypeptide, used in the methods of this invention, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated at any desired concentration, including relatively high concentrations in aqueous pharmaceutical solutions.

The dosage level for a C5 inhibitor can be any suitable level.

The plasma concentration in a patient, whether the highest level achieved or a level that is maintained, of a C5 inhibitor can be any desirable or suitable concentration. Such plasma concentration can be measured by methods known in the art. In certain embodiments, the concentration in the plasma of a patient (such as a human patient) of eculizumab or an eculizumab variant is in the range from about 25 μg/mL to about 500 μg/mL (such as between, for example, about 35 μg/mL to about 100 g/mL), Such a plasma concentration of an anti-C5 antibody, in a patient can be the highest attained after administering the anti-C5 anti body, or can be a concentration of an anti-C5 antibody in a patient that is maintained throughout the therapy. However, greater amounts (concentrations) may be required for extreme cases and smaller amounts may be sufficient for milder cases: and the amount can vary at different times during therapy. In certain embodiments, the plasma concentration of an eculizumab or an eculizumab variant can be maintained at or above about 35 μg/mL during treatment. In some embodiments, the plasma concentration of the plasma concentration of eculizumab or an eculizumab variant can be maintained at or above about 50 μg/mL during treatment.

In some embodiments, the plasma concentration of a C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be maintained at or above about 200 nM, or at or above between about 280 nM to 285 nM, during treatment.

In other treatment scenarios, the plasma concentration of eculizumab or an eculizumab variant can be maintained at or above about 75 μg/mL during treatment. In the most serious treatment scenarios, the plasma concentration of eculizumab or an eculizumab variant can be maintained at or above about 100 μg/mL during treatment.

In certain embodiments, the plasma concentration of a C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be maintained at or above about 200 nM to about 430 nM, or at or above about 570 nM to about 580 nM, during treatment.

In certain embodiments, the pharmaceutical composition is in a single unit dosage form. In certain embodiments, the single unit dosage form is between about 300 mg to about 1200 mg unit dosage form (such as about 300 mg, about 900 mg, and about 1200 mg) of a C5 inhibitor, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant. In certain embodiments, the pharmaceutical composition is lyophilized. In certain embodiments, the pharmaceutical composition is a sterile solution. In certain embodiments, the pharmaceutical composition is a preservative free formulation. In certain embodiments, the pharmaceutical composition comprises a 300 mg single-use formulation of 30 ml of a 10 mg/ml sterile, preservative free solution.

In certain embodiments, an anti-C5 full-length antibody (such as eculizumab or a variant thereof) is administered according to the following protocol: 600 mg via 25 to 45 minute IV infusion every 7+/−2 days for the first 4 weeks, followed by 900 mg for the fifth dose 7±2 days later, then 900 mg every 14±2 days thereafter. An anti-C5 antibody or polypeptide can be administered via IV infusion over 25 to 45 minute. In another embodiment, an anti-C5 polypeptide full-length antibody is administered according to the following protocol: 900 mg via 25 to 45 minute IV infusion every 7+/−2 days for the first 4 weeks, followed by 1200 mg for the fifth dose 7±2 days later, then 1200 mg every 14±2 days thereafter. An anti-C5 antibody can be administered via IV infusion over 25 to 45 minute. An exemplary pediatric dosing of, for example, an anti-C5 full-length antibody (such as eculizumab or a variant, thereof), tied to body-weight, is shown in Table 1:

TABLE 1
Exemplary dosing Recommendations in Pediatric
Patients for Full-length Antibodies

| Patient Body Weight | Induction | Maintenance |
|---|---|---|
| 40 kg and over | 900 mg weekly × | 1200 mg at week 5; then |
| | 4 doses | 1200 mg every 2 weeks |
| 30 kg to less than 40 kg | 600 mg weekly × | 900 mg at week 3; then |
| | 2 doses | 900 mg every 2 weeks |
| 20 kg to less than 30 kg | 600 mg weekly × | 600 mg at week 3; then |
| | 2 doses | 600 mg every 2 weeks |
| 10 kg to less than 20 kg | 600 mg weekly × | 300 mg at week 2; then |
| | 1 dose | 300 mg every 2 weeks |
| 20 kg to less than 30 kg | 600 mg weekly × | 600 mg at week 2; then |
| | 1 dose | 600 mg every 3 weeks |

Note that in certain other embodiments the anti-C5 polypeptides that are not full-length antibodies and are smaller than a full-length antibodies can be administered at a dosage that correspond to the same molarity as the dosage for a full-length antibody.

The aqueous solution can have a neutral pH, e.g., a pH between, e.g., about 6.5 and about 8 (e.g., between and inclusive of 7 and 8), The aqueous solution can have a pH of about any of the following: 6.6, 6.7, 6.8, 6.9, 7, 7.1, 7.2, 7.3, 7.4, 7.5, 7.6, 7.7, 7.8, 7.9, or 8.0. In some embodiments, the aqueous solution has a pH of greater than (or equal to) about 6 (e.g., greater than or equal to about any of the following: 6.1, 6.2, 6.3, 6.4, 6.5, 6.6, 6.7, 6.8, 6.9, 7, 7.1, 7.2, 7.3, 7.4, 7.5, 7.6, 7.7, 7.8, or 7.9), but less than about pH 8.

In some embodiments, the C5 inhibitor, including a polypeptide inhibitor, is administered intravenously to the subject (the term “subject” is used herein interchangeably with the term “patient”), including by intravenous injection or by intravenous infusion. In some embodiments, the anti-C5 antibody is administered intravenously to the subject, including by intravenous infusion. In some embodiments, the C5 inhibitor, including a polypeptide inhibitor, is administered to the lungs of the subject. In some embodiments, the C5 inhibitor, including a polypeptide inhibitor, is administered to the subject by subcutaneous injection. In some embodiments, the inhibitor, including a polypeptide inhibitor, is administered to the subject by-way of intraarticular injection. In some embodiments, the C5 inhibitor, including a polypeptide inhibitor, is administered to the subject by way of intravitreal or intraocular injection. In some embodiments, the inhibitor, including a polypeptide inhibitor, is administered to the subject by pulmonary delivery, such as by intrapulmonary injection (especially for pulmonary sepsis). Additional suitable routes of administration are also contemplated.

A C5 inhibitor, such as a C5-binding polypeptide, can be administered to a subject as a monotherapy. In some embodiments, the methods described herein can include administering to the subject one or more additional treatment, such as one or more additional therapeutic agents.

The additional treatment can be any additional treatment, including an experimental treatment. The other treatment can be any treatment, any therapeutic agent, which improves or stabilizes the patient's health. The additional therapeutic agent(s) includes IV fluids, such as water and/or saline, acetaminophen, heparin, one or more clotting factors, antibiotics, etc. The one or more additional therapeutic agents can be administered together with the C5 inhibitor as separate therapeutic compositions or one therapeutic composition can be formulated to include both; (i) one or more C5 inhibitors such as C5-binding polypeptides and (ii) one or more additional therapeutic agents. An additional therapeutic agent can be administered prior to, concurrently, or after administration of the C5-binding polypeptide. An additional agent and a C5 inhibitor, such as C5-binding polypeptide, can be administered using the same delivery method or route or using a different delivery method or route. The additional therapeutic agent can be another complement inhibitor, including another C5 inhibitor.

In some embodiments, an inhibitor, such as a C5-binding polypeptide, used in the methods of this invention can be formulated with one or more additional active agents.

When a C5 inhibitor is to be used in combination with a second active agent, the agents can be formulated separately or together. For example, the respective pharmaceutical compositions can be mixed, e.g., just prior to administration, and administered together or can be administered separately, e.g., at the same or different times, by the same route or different route.

In some embodiments, a composition can be formulated to include a sub-therapeutic amount of a C5 inhibitor and a sub-therapeutic amount of one or more additional active agents such that the components in total are therapeutically effective for treating a complement-associated disorder. Methods for determining a therapeutically effective dose of an agent such as a therapeutic antibody are known in the art.

The compositions can be administered to a subject, e.g., a human subject, using a variety of methods that depend, in part, on the route of administration. The route can be, e.g., intravenous (“IV”) injection or infusion, subcutaneous (“SC”) injection, intraperitoneal (“IP”) injection, pulmonary delivery such as by intrapulmonary injection (especially for pulmonary-sepsis), intraocular injection, intraarticular injection, or intramuscular (“IM”) injection.

A suitable dose of a C5 inhibitor can depend on a variety of factors including, e.g., the age, gender, and weight of a subject to be treated and the particular inhibitor compound used. Other factors affecting the dose administered to the subject include, e.g., the type or severity of the illness. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will depend upon the judgment of the treating medical practitioner (e.g., doctor or nurse).

A C5 inhibitor can be administered as a fixed dose, or in a milligram per kilogram (mg/kg) dose. In some embodiments, the dose can also be chosen to reduce or avoid production of antibodies or other host immune responses against one or more of the active antibodies in the composition.

A pharmaceutical composition can include a therapeutically effective amount of a C5inhibitor. Such effective amounts can be readily determined by one of ordinary skill in the art.

In certain embodiments, the dosing of a C5 inhibitor, such as eculizumab or a variant thereof, can be as follows: (1) administering to patient with a complement-associated disorder with about 900 milligrams (mg) of eculizumab each week for the first 3 weeks, or (2) 1200 milligrams (mg) of eculizumab each week for the first 3 weeks and (3) followed by an about 1200 mg dose on weeks 4, 6, and 8. After an initial 8-week eculizumab treatment period, the treating medical practitioner (such as a physician) can optionally request (and administer) treatment with eculizumab about 1200 mg every other week for an additional 8 weeks. The patient can then be observed for 28 weeks following eculizumab treatment.

The terms “therapeutically effective amount” or “therapeutically effective dose,” or similar terms (such as “effective amount”) used herein are intended to mean an amount of a C5 inhibitor, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, that will elicit the desired biological or medical response.

In some embodiments, a composition described herein contains a therapeutically effective amount of a C5 inhibitor, such as a C5-binding polypeptide. In some embodiments, the composition contains any C5 inhibitor, such as a C5-binding polypeptide, and one or more (e.g., one, two, three, four, five, six, seven, eight, nine, ten, or eleven or more) additional therapeutic agents such that the composition as a whole is therapeutically effective. For example, a composition can contain a C5-binding polypeptide described herein and an immunosuppressive agent, wherein the polypeptide and agent are each at a concentration that when combined are therapeutically effective for treating or preventing a complement-associated disorder in a subject.

A “subject,” as used herein, can be a human. A “patient” is used herein interchangeably with a “subject.” In certain embodiments, the patient (or the subject) is a human patient (or human subject).

### EXAMPLES

For this invention to be better understood, the following examples are set forth. These examples are for purposes of illustration only and are not be construed as limiting the scope of the invention in any manner.

### Example 1

### Eculizumab Treatment

From 1 mg per kg to 100 mg per kg per patient per treatment of a formulation comprising eculizumab (Alexion Pharmaceuticals, Inc., Cheshire Conn.) are administered to human patients diagnosed with a complement-associated disorder by intravenous infusion. Half of the patients have been vaccinated with one or more Neisseria meningococcal Type B specific vaccine, such as BEXSERO® and/or Trumenba®; the other half have not.

The patients are monitored for meningitis by methods known in the art.

### Other Embodiments

The foregoing description discloses only exemplary embodiments. It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover ail such modifications and changes as fall within the true spirit of the invention.

### Summary of Sequence Listing

| amino acid sequence of heavy chain CDR1 of | |
|---|---|
| eculizumab (as defined under combined Kabat- | |
| Chothia definition) | |
| SEQ ID NO: 1 | |
| GYIFSNYWIQ | |
| amino acid sequence of heavy chain CDR2 of | |
| eculizumab (as defined under Kabat definition) | |
| SEQ ID NO: 2 | |
| EILPGSGSTEYTENFKD | |
| amino acid sequence of the heavy chain CDR3 of | |
| eculizumab (as defined under combined Kabat | |
| definition) | |
| SEQ ID NO: 3 | |
| YFFGSSPNWYFDV | |
| amino acid sequence of the light chain CDR1 of | |
| eculizumab (as defined under Kabat definition) | |
| SEQ ID NO: 4 | |
| GASENIYGALN | |
| amino acid sequence of light chain CDR2 of | |
| eculizumab (as defined under Kabat definition) | |
| SEQ ID NO: 5 | |
| GATNLAD | |
| amino acid sequence of light chain CDR3 of | |
| eculizumab (as defined under Kabat definition) | |
| SEQ ID NO: 6 | |
| QNVLNTPLT | |
| amino acid sequence of heavy chain variable | |
| region of eculizumab | |
| SEQ ID NO: 7 | |
| QVQLVQSGAEVKKPGASVKVSCKASGYIFSNYWIQWVRQAPGQGLEW | |
| MGEILPGSGSTEYTENFKDRVTMTRDTSTSTVYMELSSLRSEDTAVY | |
| YCARYFFGSSPNWYFDVWGQGTLVTVSS | |
| amino acid sequence of light chain variable | |
| region of eculizumab, BNJ441 antibody, and | |
| BNJ421 antibody | |
| SEQ ID NO: 8 | |
| DIQMTQSPSSLSASVGDRVTITCGASENIYGALNWYQQKPGKAPKLL | |
| IYGATNLADGVPSRFSGSGSGTDFTLTISSLQPEDFATYYCQNVLNT | |
| PLTFGQGTKVEIK | |
| amino acid sequence of heavy chain constant | |
| region of eculizumab and BNJ421 antibody | |
| SEQ ID NO: 9 | |
| ASTKGPSVFPLAPCSRSTSESTAALGCLVKDYFPEPVTVSWNSGALT | |
| SGVHTFPAVLQSSGLYSLSSVVTVPSSNFGTQTYTCNVDHKPSNTKV | |
| DKTVERKCCVECPPCPAPPVAGPSVFLFPPKPKDTLMISRTPEVTCV | |
| VVDVSQEDPEVQFNWYVDGVEVHNAKTKPREEQFNSTYRVVSVLTVL | |
| HQDWLNGKEYKCKVSNKGLPSSIEKTISKAKGQPREPQVYTLPPSQE | |
| EMTKNQVSLTCLVKGFYPSDIAVEWESNGQPENNYKTTPPVLDSDGS | |
| FFLYSRLTVDKSRWQEGNVFSCSVMHEALHNHYTQKSLSLSLGK | |
| amino acid sequence of entire heavy chain of | |
| eculizumab | |
| SEQ ID NO: 10 | |
| QVQLVQSGAEVKKPGASVKVSCKASGYIFSNYWIQWVRQAPGQGLEW | |
| MGEILPGSGSTEYTENFKDRVTMTRDTSTSTVYMELSSLRSEDTAVY | |
| YCARYFFGSSPNWYFDVWGQGTLVTVSSASTKGPSVFPLAPCSRSTS | |
| ESTAALGCLVKDYFPEPVTVSWNSGALTSGVHTFPAVLQSSGLYSLS | |
| SVVTVPSSNFGTYTCNVDHKPSNTKVDKTVERKCCVECPPCPAPPVA | |
| GPSVFLFPPKPKDTLMISRTPEVTCVVVDVSQEDPEVQFNWYVDGVE | |
| VHNAKTKPREEQFNSTYRVVSVLTVLHQDWLNGKEYKCKVSNKGLPS | |
| SIEKTISKAKGQPREPQVYTLPPSQEEMTKNQVSLTCLVKGFYPSDI | |
| AVEWESNGQPENNYKTTPPVLDSDGSFFLYSRLTVDKSRWQEGNVFS | |
| CSVMHEALHNHYTQKSLSLSLGK | |
| amino acid sequence of entire light chain of | |
| eculizumab, BNJ441 antibody, and BNJ421 antibody | |
| SEQ ID NO: 11 | |
| DIQMTQSPSSLSASVGDRVTITCGASENIYGALNWYQQKPGKAPKLL | |
| IYGATNLADGVPSRFSGSGSGTDFTLTISSLQPEDFATYYCQNVLNT | |
| PLTFGQGTKVEIKRTVAAPSVFIFPPSDEQLKSGTASVVCLLNNFYP | |
| REAKVQWKVDNALQSGNSQESVTEQDSKDSTYSLSSTLTLSKADYEK | |
| HKVYACEVTHQGLSSPVTKSFNRGEC | |
| amino acid sequence of heavy chain variable | |
| region of BNJ441 antibody and BNJ421 antibody | |
| SEQ ID NO: 12 | |
| QVQLVQSGAEVKKPGASVKVSCKASGHIFSNYWIQWVRQAPGQGLEW | |
| MGEILPGSGHTEYTENFKDRVTMTRDTSTSTVYMELSSLRSEDTAVY | |
| YCARYFFGSSPNWYFDVWGQGTLVTVSS | |
| amino acid sequence of heavy chain constant | |
| region of BNJ441 antibody | |
| SEQ ID NO: 13 | |
| ASTKGPSVFPLAPCSRSTSESTAALGCLVKDYFPEPVTVSWNSGALT | |
| SGVHTFPAVLQSSGLYSLSSVVTVPSSNFGTQTYTCNVDHKPSNTKV | |
| DKTVERKCCVECPPCPAPPVAGPSVFLFPPKPKDTLMISRTPEVTCV | |
| VVDVSQEDPEVQFNWYVDGVEVHNAKTKPREEQFNSTYRVVSVLTVL | |
| HQDWLNGKEYKCKVSNKGLPSSIEKTISKAKGQPREPQVYTLPPSQE | |
| EMTKNQVSLTCLVKGFYPSDIAVEWESNGQPENNYKTTPPVLDSDGS | |
| FFLYSRLTVDKSRWQEGNVFSCSVLHEALHSHYTQKSLSLSLGK | |
| amino acid sequence of entire heavy chain of | |
| BNJ441 antibody | |
| SEQ ID NO: 14 | |
| QVQLVQSGAEVKKPGASVKVSCKASGHIFSNYWIQWVRQAPGQGLEW | |
| MGEILPGSGHTEYTENFKDRVTMTRDTSTSTVYMELSSLRSEDTAVY | |
| YCARYFFGSSPNWYFDVWGQGTLVTVSSASTKGPSVFPLAPCSRSTS | |
| ESTAALGCLVKDYFPEPVTVSWNSGALTSGVHTFPAVLQSSGLYSLS | |
| SVVTVPSSNFGTQTYTCNVDHKPSNTKVDKTVERKCCVECPPCPAPP | |
| VAGPSVFLFPPKPKDTLMISRTPEVTCVVVDVSQEDPEVQFNWYVDG | |
| VEVHNAKTKPREEQFNSTYRVVSVLTVLHQDWLNGKEYKCKVSNKGL | |
| PSSIEKTISKAKGQPREPQVYTLPPSQEEMTKNQVSLTCLVKGFYPS | |
| DIAVEWESNGQPENNYKTTPPVLDSDGSFFLYSRLTVDKSRWQEGNV | |
| FSCSV [symbol] HEALH [symbol] HYTQKSLSLSLGK | |
| amino acid sequence of IgG2 heavy chain | |
| constant region variant comprising YTE | |
| substitutions | |
| SEQ ID NO: 15 | |
| ASTKGPSVFPLAPCSRSTSESTAALGCLVKDYFPEPVTVSWNSGALT | |
| SGVHTFPAVLQSSGLYSLSSVVTVTSSNFGTQTYTCNVDHKPSNTKV | |
| DKTVERKCCVECPPCPAPPVAGPSVFLFPPKPKDTLYITREPEVTCV | |
| VVDVSHEDPEVQFNWYVDGMEVHNAKTKPREEQFNSTFRVVSVLTVV | |
| HQDWLNGKEYKCKVSNKGLPAPIEKTISKTKGQPREPQVYTLPPSRE | |
| EMTKNQVSLTCLVKGFYPSDIAVEWESNGQPENNYKTTPPMLDSDGS | |
| FFLYSKLTVDKSRWQQGNVFSCSVMHEALHNHYTQKSLSLSPGK | |
| amino acid sequence of entire heavy chain of | |
| eculizumab variant comprising heavy chain | |
| constant region depicted in SEQ ID NO: 15 | |
| (above) | |
| SEQ ID NO: 16 | |
| QVQLVQSGAEVKKPGASVKVSCKASGYIFSNYWIQWVRQAPGQGLEW | |
| MGEILPGSGSTEYTENFKDRVTMTRDTSTSTVYMELSSLRSEDTAVY | |
| YCARYFFGSSPNWYFDVWGQGTLVTVSSASTKGPSVFPLAPCSRSTS | |
| ESTAALGCLVKDYFPEPVTVSWNSGALTSGVHTFPAVLQSSGLYSLS | |
| SVVTVTSSNFGTQTYTCNVDHKPSNTKVDKTVERKCCVECPPCPAPP | |
| VAGPSVFLFPPKPKDTLYITREPEVTCVVVDVSHEDPEVQFNWYVDG | |
| MEVHNAKTKPREEQFNSTFRVVSVLTVVHQDWLNGKEYKCKVSNKGL | |
| PAPIEKTISKTKGQPREPQVYTLPPSREEMTKNQVSLTCLVKGFYPS | |
| DIAVEWESNGQPENNYKTTPPMLDSDGSFFLYSKLTVDKSRWQQGNV | |
| FSCSVMHEALHNHYTQKSLSLSPGK | |
| amino acid sequence of light chain CDR1 of | |
| eculizumab (as defined under Kabat definition) | |
| wi glycine to histidine substitution at | |
| position 8 relative to SEQ ID NO: 4 | |
| SEQ ID NO: 17 | |
| GASENIYHALN | |
| depicts amino acid sequence of heavy chain CDR2 | |
| of eculizumab in which serine at position 8 | |
| relative to SEQ ID NO: 2 is substituted with | |
| histidine | |
| SEQ ID NO: 18 | |
| EILPGSGHTEYTENFKD | |
| amino acid sequence of heavy chain CDR1 of | |
| eculizumab in which tyrosine at position 2 | |
| (relative to SEQ ID NO: 1) is substituted with | |
| histidine | |
| SEQ ID NO: 19 | |
| GHIFSNYWIQ | |
| amino acid sequence of entire heavy chain of | |
| BNJ421 antibody | |
| SEQ ID NO: 20 | |
| QVQLVQSGAEVKKPGASVKVSCKASGHIFSNYWIQWVRQAPGQGLEW | |
| MGEILPGSGHTEYTENFKDRVTMTRDTSTSTVYMELSSLRSEDTAVY | |
| YCARYFFGSSPNWYFDVWGQGTLVTVSSASTKGPSVFPLAPCSRSTS | |
| ESTAALGCLVKDYFPEPVTVSWNSGALTSGVHTFPAVLQSSGLYSLS | |
| SVVTVPSSNFGTQTYTCNVDHKPSNTKVDKTVERKCCVECPPCPAPP | |
| VAGPSVFLFPPKPKDTLMISRTPEVTCVVVDVSQEDPEVQFNWYVDG | |
| VEVHNAKTKPREEQFNSTYRVVSVLTVLHQDWLNGKEYKCKVSNKGL | |
| PSSIEKTISKAKGQPREPQVYTLPPSQEEMTKNQVSLTCLVKGFYPS | |
| DIAVEWESNGQPENNYKTTPPVLDSDGSFFLYSRLTVDKSRWQEGNV | |
| FSCSV [symbol] HEAL [symbol] NHYTQKSLSLSLGK | |
| single chain antibody pexelizumab | |
| SEQ ID NO: 21 | |
| gat atc cag atg acc cag tcc ccg tcc tcc ctg tcc gcc tct gtg ggc | 48 |
| Asp Ile Gln Met Thr Gln Ser Pro Ser Ser Leu Ser Ala Ser Val Gly | |
| 1 5 10 15 | |
| gat agg gtc acc atc acc tgc ggc gcc agc gaa aac atc tat ggc gcg | 96 |
| Asp Arg Val Thr Ile Thr Cys Gly Ala Ser Glu Asn Ile Tyr Gly Ala | |
| 20 25 30 | |
| ctg aac tgg tat caa cag aaa ccc ggg aaa gct ccg aag ctt ctg att | 144 |
| Leu Asn Trp Tyr Gln Gln Lys Pro Gly Lys Ala Pro Lys Leu Leu Ile | |
| 35 40 45 | |
| tac ggt gcg acg aac ctg gca gat gga gtc cct tct cgc ttc tct gga | 192 |
| Tyr Gly Ala Thr Asn Leu Ala Asp Gly Val Pro Ser Arg Phe Ser Gly | |
| 50 55 60 | |
| tcc ggc tcc gga acg gat ttc act ctg acc atc agc agt ctg cag cct | 240 |
| Ser Gly Ser Gly Thr Asp Phe Thr Leu Thr Ile Ser Ser Leu Gln Pro | |
| 65 70 75 80 | |
| gaa gac ttc gct acg tat tac tgt cag aac gtt tta aat act ccg ttg | 288 |
| Glu Asp Phe Ala Thr Tyr Tyr Cys Gln Asn Val Leu Asn Thr Pro Leu | |
| 85 90 95 | |
| act ttc gga cag ggt acc aag gtg gaa ata aaa cgt act ggc ggt ggt | 336 |
| Thr Phe Gly Gln Gly Thr Lys Val Glu Ile Lys Arg Thr Gly Gly Gly | |
| 100 105 110 | |
| ggt tct ggt ggc ggt gga tct ggt ggt ggc ggt tct caa gtc caa ctg | 384 |
| Gly Ser Gly Gly Gly Gly Ser Gly Gly Gly Gly Ser Gln Val Gln Leu | |
| 115 120 125 | |
| gtg caa tcc ggc gcc gag gtc aag aag cca ggg gcc tca gtc aaa gtg | 432 |
| Val Gln Ser Gly Ala Glu Val Lys Lys Pro Gly Ala Ser Val Lys Val | |
| 130 135 140 | |
| tcc tgt aaa gct agc ggc tat att ttt tct aat tat tgg att caa tgg | 480 |
| Ser Cys Lys Ala Ser Gly Tyr Ile Phe Ser Asn Tyr Trp Ile Gln Trp | |
| 145 150 155 160 | |
| gtg cgt cag gcc ccc ggg cag ggc ctg gaa tgg atg ggt gag atc tta | 528 |
| Val Arg Gln Ala Pro Gly Gln Gly Leu Glu Trp Met Gly Glu Ile Leu | |
| 165 170 175 | |
| ccg ggc tct ggt agc acc gaa tat acc gaa aat ttt aaa gac cgt gtt | 576 |
| Pro Gly Ser Gly Ser Thr Glu Tyr Thr Glu Asn Phe Lys Asp Arg Val | |
| 180 185 190 | |
| act atg acg cgt gac act tcg act agt aca gta tac atg gag ctc tcc | 624 |
| Thr Met Thr Arg Asp Thr Ser Thr Ser Thr Val Tyr Met Glu Leu Ser | |
| 195 200 205 | |
| agc ctg cga tcg gag gac acg gcc gtc tat tat tgc gcg cgt tat ttt | 672 |
| Ser Leu Arg Ser Glu Asp Thr Ala Val Tyr Tyr Cys Ala Arg Tyr Phe | |
| 210 215 220 | |
| ttt ggt tct agc ccg aat tgg tat ttt gat gtt tgg ggt caa gga acc | 720 |
| Phe Gly Ser Ser Pro Asn Trp Tyr Phe Asp Val Trp Gly Gln Gly Thr | |
| 225 230 235 240 | |
| ctg gtc act gtc tcg age tga | 741 |
| Leu Val Thr Val Ser Ser | |
| 245 | |

```
variant of pexelizumab in which the arginine
(R) at position 38 has been substituted with
glutamine (Q)
SEQ ID NO: 22
Asp Ile Gln Met Thr Gln Ser Pro Ser Ser Leu Ser Ala Ser Val Gly
    1 5 10 15
Asp Arg Val Thr Ile Thr Cys Gly Ala Ser Glu Asn Ile Tyr Gly Ala
                          20 25 30
Leu Asn Trp Tyr Gln Gln Lys Pro Gly Lys Ala Pro Lys Leu Leu Ile
                  35 40 45
Tyr Gly Ala Thr Asn Leu Ala Asp Gly Val Pro Ser Arg Phe Ser Gly
          50 55 60
Ser Gly Ser Gly Thr Asp Phe Thr Leu Thr Ile Ser Ser Leu Gln Pro
  65 70 75 80
Glu Asp Phe Ala Thr Tyr Tyr Cys Gln Asn Val Leu Asn Thr Pro Leu
                                  85 90 95
Thr Phe Gly Gln Gly Thr Lys Val Glu Ile Lys Arg Thr Gly Gly Gly
                        100 105 110
Gly Ser Gly Gly Gly Gly Ser Gly Gly Gly Gly Ser Gln Val Gln Leu
                115 120 125
Val Gln Ser Gly Ala Glu Val Lys Lys Pro Gly Ala Ser Val Lys Val
        130 135 140
Ser Cys Lys Ala Ser Gly Tyr Ile Phe Ser Asn Tyr Trp Ile Gln Trp
145 150 155 160
Val Arg Gln Ala Pro Gly Gln Gly Leu Glu Trp Met Gly Glu Ile Leu
                                165 170 175
Pro Gly Ser Gly Ser Thr Glu Tyr Thr Glu Asn Phe Lys Asp Arg Val
                        180 185 190
Thr Met Thr Arg Asp Thr Ser Thr Ser Thr Val Tyr Met Glu Leu Ser
                195 200 205
Ser Leu Arg Ser Glu Asp Thr Ala Val Tyr Tyr Cys Ala Arg Tyr Phe
        210 215 220
Phe Gly Ser Ser Pro Asn Trp Tyr Phe Asp Val Trp Gly Gln Gly Thr
225 230 235 240
Leu Val Thr Val Ser Ser
                                245
exemplary linker amino acid sequence present in a
variant pexelizumab antibody is shown in SEQ ID NO: 3
SEQ ID NO: 23
Gly Gly Gly Gly Ser Gly Gly Gly Gly Ser Gly Gly Gly Gly Ser
SEQ ID NO: 24
Met Gly Leu Leu Gly Ile Leu Cys Phe Leu Ile Phe Leu Gly Lys Thr
    1 5 10 15
Trp Gly Gln Glu Gln Thr Tyr Val Ile Ser Ala Pro Lys Ile Phe Arg
                          20 25 30
Val Gly Ala Ser Glu Asn Ile Val Ile Gln Val Tyr Gly Tyr Thr Glu
                  35 40 45
Ala Phe Asp Ala Thr Ile Ser Ile Lys Ser Tyr Pro Asp Lys Lys Phe
          50 55 60
Ser Tyr Ser Ser Gly His Val His Leu Ser Ser Glu Asn Lys Phe Gln
65 70 75 80
Asn Ser Ala Ile Leu Thr Ile Gln Pro Lys Gln Leu Pro Gly Gly Gln
                                  85 90 95
Asn Pro Val Ser Tyr Val Tyr Leu Glu Val Val Ser Lys His Phe Ser
                        100 105 110
Lys Ser Lys Arg Met Pro Ile Thr Tyr Asp Asn Gly Phe Leu Phe Ile
                115 120 125
His Thr Asp Lys Pro Val Tyr Thr Pro Asp Gln Ser Val Lys Val Arg
        130 135 140
Val Tyr Ser Leu Asn Asp Asp Leu Lys Pro Ala Lys Arg Glu Thr Val
145 150 155 160
Leu Thr Phe Ile Asp Pro Glu Gly Ser Glu Val Asp Met Val Glu Glu
                                165 170 175
Ile Asp His Ile Gly Ile Ile Ser Phe Pro Asp Phe Lys Ile Pro Ser
                        180 185 190
Asn Pro Arg Tyr Gly Met Trp Thr Ile Lys Ala Lys Tyr Lys Glu Asp
                195 200 205
Phe Ser Thr Thr Gly Thr Ala Tyr Phe Glu Val Lys Glu Tyr Val Leu
        210 215 220
Pro His Phe Ser Val Ser Ile Glu Pro Glu Tyr Asn Phe Ile Gly Tyr
225 230 235 240
Lys Asn Phe Lys Asn Phe Glu Ile Thr Ile Lys Ala Arg Tyr Phe Tyr
                                245 250 255
Asn Lys Val Val Thr Glu Ala Asp Val Tyr Ile Thr Phe Gly Ile Arg
                        260 265 270
Glu Asp Leu Lys Asp Asp Gln Lys Glu Met Met Gln Thr Ala Met Gln
                275 280 285
Asn Thr Met Leu Ile Asn Gly Ile Ala Gln Val Thr Phe Asp Ser Glu
        290 295 300
Thr Ala Val Lys Glu Leu Ser Tyr Tyr Ser Leu Glu Asp Leu Asn Asn
305 310 315 320
Lys Tyr Leu Tyr Ile Ala Val Thr Val Ile Glu Ser Thr Gly Gly Phe
                                325 330 335
Ser Glu Glu Ala Glu Ile Pro Gly Ile Lys Tyr Val Leu Ser Pro Tyr
                        340 345 350
Lys Leu Asn Leu Val Ala Thr Pro Leu Phe Leu Lys Pro Gly Ile Pro
                355 360 365
Tyr Pro Ile Lys Val Gln Val Lys Asp Ser Leu Asp Gln Leu Val Gly
        370 375 380
Gly Val Pro Val Ile Leu Asn Ala Gln Thr Ile Asp Val Asn Gln Glu
385 390 395 400
Thr Ser Asp Leu Asp Pro Ser Lys Ser Val Thr Arg Val Asp Asp Gly
                                405 410 415
Val Ala Ser Phe Val Leu Asn Leu Pro Ser Gly Val Thr Val Leu Glu
                        420 425 430
Phe Asn Val Lys Thr Asp Ala Pro Asp Leu Pro Glu Glu Asn Gln Ala
                435 440 445
Arg Glu Gly Tyr Arg Ala Ile Ala Tyr Ser Ser Leu Ser Gln Ser Tyr
        450 455 460
Leu Tyr Ile Asp Trp Thr Asp Asn His Lys Ala Leu Leu Val Gly Glu
465 470 475 480
His Leu Asn Ile Ile Val Thr Pro Lys Ser Pro Tyr Ile Asp Lys Ile
                                485 490 495
Thr His Tyr Asn Tyr Leu Ile Leu Ser Lys Gly Lys Ile Ile His Phe
                        500 505 510
Gly Thr Arg Glu Lys Phe Ser Asp Ala Ser Tyr Gln Ser Ile Asn Ile
                515 520 525
Pro Val Thr Gln Asn Met Val Pro Ser Ser Arg Leu Leu Val Tyr Tyr
        530 535 540
Ile Val Thr Gly Glu Gln Thr Ala Glu Leu Val Ser Asp Ser Val Trp
545 550 555 560
Leu Asn Ile Glu Glu Lys Cys Gly Asn Gln Leu Gln Val His Leu Ser
                                565 570 575
Pro Asp Ala Asp Ala Tyr Ser Pro Gly Gln Thr Val Ser Leu Asn Met
                        580 585 590
Ala Thr Gly Met Asp Ser Trp Val Ala Leu Ala Ala Val Asp Ser Ala
                595 600 605
Val Tyr Gly Val Gln Arg Gly Ala Lys Lys Pro Leu Glu Arg Val Phe
        610 615 620
Gln Phe Leu Glu Lys Ser Asp Leu Gly Cys Gly Ala Gly Gly Gly Leu
625 630 635 640
Asn Asn Ala Asn Val Phe His Leu Ala Gly Leu Thr Phe Leu Thr Asn
                                645 650 655
Ala Asn Ala Asp Asp Ser Gln Glu Asn Asp Glu Pro Cys Lys Glu Ile
                        660 665 670
Leu Arg Pro Arg Arg Thr Leu Gln Lys Lys Ile Glu Glu Ile Ala Ala
                675 680 685
Lys Tyr Lys His Ser Val Val Lys Lys Cys Cys Tyr Asp Gly Ala Cys
        690 695 700
Val Asn Asn Asp Glu Thr Cys Glu Gln Arg Ala Ala Arg Ile Ser Leu
705 710 715 720
Gly Pro Arg Cys Ile Lys Ala Phe Thr Glu Cys Cys Val Val Ala Ser
                                725 730 735
Gln Leu Arg Ala Asn Ile Ser His Lys Asp Met Gln Leu Gly Arg Leu
                        740 745 750
His Met Lys Thr Leu Leu Pro Val Ser Lys Pro Glu Ile Arg Ser Tyr
                755 760 765
Phe Pro Glu Ser Trp Leu Trp Glu Val His Leu Val Pro Arg Arg Lys
        770 775 780
Gln Leu Gln Phe Ala Leu Pro Asp Ser Leu Thr Thr Trp Glu Ile Gln
785 790 795 800
Gly Ile Gly Ile Ser Asn Thr Gly Ile Cys Val Ala Asp Thr Val Lys
                                805 810 815
Ala Lys Val Phe Lys Asp Val Phe Leu Glu Met Asn Ile Pro Tyr Ser
                        820 825 830
Val Val Arg Gly Glu Gln Ile Gln Leu Lys Gly Thr Val Tyr Asn Tyr
                835 840 845
Arg Thr Ser Gly Met Gln Phe Cys Val Lys Met Ser Ala Val Glu Gly
        850 855 860
Ile Cys Thr Ser Glu Ser Pro Val Ile Asp His Gln Gly Thr Lys Ser
865 870 875 880
Ser Lys Cys Val Arg Gln Lys Val Glu Gly Ser Ser Ser His Leu Val
                                885 890 895
Thr Phe Thr Val Leu Pro Leu Glu Ile Gly Leu His Asn Ile Asn Phe
                        900 905 910
Ser Leu Glu Thr Trp Phe Gly Lys Glu Ile Leu Val Lys Thr Leu Arg
                915 920 925
Val Val Pro Glu Gly Val Lys Arg Glu Ser Tyr Ser Gly Val Thr Leu
        930 935 940
Asp Pro Arg Gly Ile Tyr Gly Thr Ile Ser Arg Arg Lys Glu Phe Pro
945 950 955 960
Tyr Arg Ile Pro Leu Asp Leu Val Pro Lys Thr Glu Ile Lys Arg Ile
                                965 970 975
Leu Ser Val Lys Gly Leu Leu Val Gly Glu Ile Leu Ser Ala Val Leu
                        980 985 990
Ser Gln Glu Gly Ile Asn Ile Leu Thr His Leu Pro Lys Gly Ser Ala
                995 1000 1005
Glu Ala Glu Leu Met Ser Val Val Pro Val Phe Tyr Val Phe His
      1010 1015 1020
Tyr Leu Glu Thr Gly Asn His Trp Asn Ile Phe His Ser Asp Pro
      1025 1030 1035
Leu Ile Glu Lys Gln Lys Leu Lys Lys Lys Leu Lys Glu Gly Met
      1040 1045 1050
Leu Ser Ile Met Ser Tyr Arg Asn Ala Asp Tyr Ser Tyr Ser Val
      1055 1060 1065
Trp Lys Gly Gly Ser Ala Ser Thr Trp Leu Thr Ala Phe Ala Leu
      1070 1075 1080
Arg Val Leu Gly Gln Val Asn Lys Tyr Val Glu Gln Asn Gln Asn
      1085 1090 1095
Ser Ile Cys Asn Ser Leu Leu Trp Leu Val Glu Asn Tyr Gln Leu
      1100 1105 1110
Asp Asn Gly Ser Phe Lys Glu Asn Ser Gln Tyr Gln Pro Ile Lys
      1115 1120 1125
Leu Gln Gly Thr Leu Pro Val Glu Ala Arg Glu Asn Ser Leu Tyr
      1130 1135 1140
Leu Thr Ala Phe Thr Val Ile Gly Ile Arg Lys Ala Phe Asp Ile
      1145 1150 1155
Cys Pro Leu Val Lys Ile Asp Thr Ala Leu Ile Lys Ala Asp Asn
      1160 1165 1170
Phe Leu Leu Glu Asn Thr Leu Pro Ala Gln Ser Thr Phe Thr Leu
      1175 1180 1185
Ala Ile Ser Ala Tyr Ala Leu Ser Leu Gly Asp Lys Thr His Pro
      1190 1195 1200
Gln Phe Arg Ser Ile Val Ser Ala Leu Lys Arg Glu Ala Leu Val
      1205 1210 1215
Lys Gly Asn Pro Pro Ile Tyr Arg Phe Trp Lys Asp Asn Leu Gln
      1220 1225 1230
His Lys Asp Ser Ser Val Pro Asn Thr Gly Thr Ala Arg Met Val
      1235 1240 1245
Glu Thr Thr Ala Tyr Ala Leu Leu Thr Ser Leu Asn Leu Lys Asp
      1250 1255 1260
Ile Asn Tyr Val Asn Pro Val Ile Lys Trp Leu Ser Glu Glu Gln
      1265 1270 1275
Arg Tyr Gly Gly Gly Phe Tyr Ser Thr Gln Asp Thr Ile Asn Ala
      1280 1285 1290
Ile Glu Gly Leu Thr Glu Tyr Ser Leu Leu Val Lys Gln Leu Arg
      1295 1300 1305
Leu Ser Met Asp Ile Asp Val Ser Tyr Lys His Lys Gly Ala Leu
      1310 1315 1320
His Asn Tyr Lys Met Thr Asp Lys Asn Phe Leu Gly Arg Pro Val
      1325 1330 1335
Glu Val Leu Leu Asn Asp Asp Leu Ile Val Ser Thr Gly Phe Gly
      1340 1345 1350
Ser Gly Leu Ala Thr Val His Val Thr Thr Val Val His Lys Thr
      1355 1360 1365
Ser Thr Ser Glu Glu Val Cys Ser Phe Tyr Leu Lys Ile Asp Thr
      1370 1375 1380
Gln Asp Ile Glu Ala Ser His Tyr Arg Gly Tyr Gly Asn Ser Asp
      1385 1390 1395
Tyr Lys Arg Ile Val Ala Cys Ala Ser Tyr Lys Pro Ser Arg Glu
      1400 1405 1410
Glu Ser Ser Ser Gly Ser Ser His Ala Val Met Asp Ile Ser Leu
      1415 1420 1425
Pro Thr Gly Ile Ser Ala Asn Glu Glu Asp Leu Lys Ala Leu Val
      1430 1435 1440
Glu Gly Val Asp Gln Leu Phe Thr Asp Tyr Gln Ile Lys Asp Gly
      1445 1450 1455
His Val Ile Leu Gln Leu Asn Ser Ile Pro Ser Ser Asp Phe Leu
      1460 1465 1470
Cys Val Arg Phe Arg Ile Phe Glu Leu Phe Glu Val Gly Phe Leu
      1475 1480 1485
Ser Pro Ala Thr Phe Thr Val Tyr Glu Tyr His Arg Pro Asp Lys
      1490 1495 1500
Gln Cys Thr Met Phe Tyr Ser Thr Ser Asn Ile Lys Ile Gln Lys
      1505 1510 1515
Val Cys Glu Gly Ala Ala Cys Lys Cys Val Glu Ala Asp Cys Gly
      1520 1525 1530
Gln Met Gln Glu Glu Leu Asp Leu Thr Ile Ser Ala Glu Thr Arg
      1535 1540 1545
Lys Gln Thr Ala Cys Lys Pro Glu Ile Ala Tyr Ala Tyr Lys Val
      1550 1555 1560
Ser Ile Thr Ser Ile Thr Val Glu Asn Val Phe Val Lys Tyr Lys
      1565 1570 1575
Ala Thr Leu Leu Asp Ile Tyr Lys Thr Gly Glu Ala Val Ala Glu
      1580 1585 1590
Lys Asp Ser Glu Ile Thr Phe Ile Lys Lys Val Thr Cys Thr Asn
      1595 1600 1605
Ala Glu Leu Val Lys Gly Arg Gln Tyr Leu Ile Met Gly Lys Glu
      1610 1615 1620
Ala Leu Gln Ile Lys Tyr Asn Phe Ser Phe Arg Tyr Ile Tyr Pro
      1625 1630 1635
Leu Asp Ser Leu Thr Trp Ile Glu Tyr Trp Pro Arg Asp Thr Thr
      1640 1645 1650
Cys Ser Ser Cys Gln Ala Phe Leu Ala Asn Leu Asp Glu Phe Ala
      1655 1660 1665
Glu Asp Ile Phe Leu Asn Gly Cys
      1670 1675
SEQ ID NO: 25
QVQLVQSGAEVKKPGASVKVSCKASGYIFSNYWIQWVRQAPGQGLEWMGEILP
GSGSTEYTENFKDRVTMTRDTSTSTVYMELSSLRSEDTAVYYCARYFFGSSPN
WYFDVWGQGTLVTVSSASTKGPSVFPLAPCSRSTSESTAALGCLVKDYFPEPV
TVSWNSGALTSGVHTFPAVLQSSGLYSLSSVVTVPSSNFGTQTYTCNVDHKPS
NTKVDKTVERKCCVECPPCPAPPVAGPSVFLFPPKPKDTLMISRTPEVTCVVV
DVSQEDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVF
SCSVMHEALHNHYTQKSLSLSLGK
SEQ ID NO: 26
DIQMTQSPSSLSASVGDRVTITCGASENIYGALNWYQQKPGKAPKLLIYGATN
LADGVPSRFSGSGSGTDFTLTISSLQPEDFATYYCQNVLNTPLTFGQGTKVEI
KRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRG
EC
heavy chain (g_{2/4}) (448 amino acids)
SEQ ID NO: 27
QVQLVQSGAEVKKPGASVKVSCKASGHIFSNYWIQWVRQAPGQGLEWMGEILP
GSGHTEYTENFKDRVTMTRDTSTSTVYMELSSLRSEDTAVYYCARYFFGSSPN
WYFDVWGQGTLVTVSSASTKGPSVFPLAPCSRSTSESTAALGCLVKDYFPEPV
TVSWNSGALTSGVHTFPAVLQSSGLYSLSSVVTVPSSNFGTQTYTCNVDHKPS
NTKVDKTVERKCCVECPPCPAPPVAGPSVFLFPPKPKDTLMISRTPEVTCVVV
DVSQEDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVF
SCSVLHEALHSHYTQKSLSILGK
```

1. A method of treating a human patient in need of treatment with eculizumab or an eculizumab variant, comprising administering an effective amount of eculizumab or an eculizumab variant to the patient, wherein the patient is one:
who has been vaccinated with a Neisseria meningococcal type B specific vaccine before the patient's treatment with eculizumab or an eculizumab variant; or
who is vaccinated with a Neisseria meningococcal type B specific vaccine concurrently with the patient's first administration with eculizumab or an eculizumab variant; or
who has been administered eculizumab or an eculizumab variant before being vaccinated with a Neisseria meningococcal type B specific vaccine and the patient is vaccinated with a Neisseria meningococcal type B specific vaccine immediately upon discovery that the patient has not been vaccinated with a Neisseria meningococcal type B specific vaccine; or
who has been administered eculizumab or an eculizumab variant before being vaccinated with a Neisseria meningococcal type B specific vaccine and that administration is interrupted until the patient is vaccinated with a Neisseria meningococcal type B specific vaccine.

2. The method of claim 1, wherein the patient in need of treatment has a complement-associated disorder.

3. The method of claim 2, wherein the patient has been diagnosed with paroxysmal nocturnal hemoglobinuria (“PNH”), atypical hemolytic uremic syndrome (“aHUS”), or Shiga-toxin-producing E. coli hemolytic uremic syndrome (“STEC-HUS”).

4. The method of claim 2, wherein the complement-associated disorder is selected from the group consisting of age-related macular degeneration, graft rejection, bone marrow rejection, kidney graft rejection, skin graft rejection, heart graft rejection, lung graft rejection, liver graft rejection, rheumatoid arthritis, a pulmonary condition, ischemia-reperfusion injury, atypical hemolytic uremic syndrome, thrombotic thrombocytopenic purpura, paroxysmal nocturnal hemoglobinuria, dense deposit disease, age-related macular degeneration, spontaneous fetal loss, Pauci-immune vasculitis, epidermolysis bullosa, recurrent fetal loss, multiple sclerosis, traumatic brain injury, myasthenia gravis, cold agglutinin disease, dermatomyositis, Degos' disease, Graves' disease, Hashimoto's thyroiditis, type I diabetes, psoriasis, pemphigus, autoimmune hemolytic anemia, idiopathic thrombocytopenic purpura, Goodpasture syndrome, multifocal motor neuropathy, neuromyelitis optica, antiphospholipid syndrome, sepsis, Hemorrhagic fever, and catastrophic antiphospholipid syndrome.

5. The method of claim 1, wherein the patient is vaccinated with a Neisseria meningococcal type B specific vaccine before the patient's treatment with eculizumab or an eculizumab variant.

6. The method of claim 1, wherein the patient is vaccinated with a Neisseria meningococcal type B specific vaccine concurrently with the patient's first administration with eculizumab or an eculizumab variant.

7. The method of claim 1, wherein the patient has been administered eculizumab or an eculizumab variant before being vaccinated with a Neisseria meningococcal type B specific vaccine and the patient is vaccinated with a Neisseria meningococcal type B specific vaccine immediately upon discovery that the patient has not been vaccinated with a Neisseria meningococcal type B specific vaccine.

8. The method of claim 1, wherein the patient has been administered eculizumab or an eculizumab variant before being vaccinated with a Neisseria meningococcal type B specific vaccine and that administration is interrupted until the patient is vaccinated with a Neisseria meningococcal type B specific vaccine.

9. The method of claim 1, wherein the Neisseria meningococcal type B specific vaccine is multicomponent meningococcal serogroup B vaccine (4CMenB) or meningococcal group B vaccine (Neisseria meningitidis serogroup B recombinant 1p2086 a05 protein variant antigen and Neisseria meningitidis serogroup B recombinant 1p2086 b01 protein variant antigen).

10. A method for inhibiting formation of terminal complement in a patient, the method comprising administering to said patient an eculizumab or an eculizumab variant in an amount effective to inhibit terminal complement in the patient, wherein the patient is one: who has been vaccinated with a Neisseria meningococcal type B specific vaccine before the patient's treatment with eculizumab or an eculizumab variant; or
who is vaccinated with a Neisseria meningococcal type B specific vaccine concurrently with the patient's first administration with eculizumab or an eculizumab variant; or
who has been administered eculizumab or an eculizumab variant before being vaccinated with a Neisseria meningococcal type B specific vaccine and the patient is vaccinated with a Neisseria meningococcal type B specific vaccine immediately upon discovery that the patient has not been vaccinated with a Neisseria meningococcal type B specific vaccine; or
who has been administered eculizumab or an eculizumab variant before being vaccinated with a Neisseria meningococcal type B specific vaccine and that administration is interrupted until the patient is vaccinated with a Neisseria meningococcal type B specific vaccine.

11. The method of claim 10, wherein the patient in need of treatment has a complement-associated disorder.

12. The method of claim 11, wherein the patient has been diagnosed with paroxysmal nocturnal hemoglobinuria (“PNH”), atypical hemolytic uremic syndrome (“aHUS”), or Shiga-toxin-producing E. coli hemolytic uremic syndrome (“STEC-HUS”).

13. The method of claim 11, wherein the complement-associated disorder is selected from the group consisting of age-related macular degeneration, graft rejection, bone marrow rejection, kidney graft rejection, skin graft rejection, heart graft rejection, lung graft rejection, liver graft rejection, rheumatoid arthritis, a pulmonary condition, ischemia-reperfusion injury, atypical hemolytic uremic syndrome, thrombotic thrombocytopenic purpura, paroxysmal nocturnal hemoglobinuria, dense deposit disease, age-related macular degeneration, spontaneous fetal loss, Pauci-immune vasculitis, epidermolysis bullosa, recurrent fetal loss, multiple sclerosis, traumatic brain injury, myasthenia gravis, cold agglutinin disease, dermatomyositis, Degos' disease, Graves' disease, Hashimoto's thyroiditis, type I diabetes, psoriasis, pemphigus, autoimmune hemolytic anemia, idiopathic thrombocytopenic purpura, Goodpasture syndrome, multifocal motor neuropathy, neuromyelitis optica, antiphospholipid syndrome, sepsis, Hemorrhagic fever, and catastrophic antiphospholipid syndrome.

14. The method of claim 10, wherein the patient is vaccinated with a Neisseria meningococcal type B specific vaccine before the patient's treatment with eculizumab or an eculizumab variant.

15. The method of claim 10, wherein the patient is vaccinated with a Neisseria meningococcal type B specific vaccine concurrently with the patient's first administration with eculizumab or an eculizumab variant.

16. The method of claim 10, wherein the patient has been administered eculizumab or an eculizumab variant before being vaccinated with a Neisseria meningococcal type B specific vaccine and the patient is vaccinated with a Neisseria meningococcal type B specific vaccine immediately upon discovery that the patient has not been vaccinated with a Neisseria meningococcal type B specific vaccine.

17. The method of claim 10, wherein the patient has been administered eculizumab or an eculizumab variant before being vaccinated with a Neisseria meningococcal type B specific vaccine and that administration is interrupted until the patient is vaccinated with a Neisseria meningococcal type B specific vaccine.

18. The method of claim 10, wherein the Neisseria meningococcal type B specific vaccine is multicomponent meningococcal serogroup B vaccine (4CMenB) or meningococcal group B vaccine (Neisseria meningitidis serogroup B recombinant 1p2086 a05 protein variant antigen and Neisseria meningitidis serogroup B recombinant 1p2086 b01 protein variant antigen).

19. A method of vaccinating a patient being treated with eculizumab or an eculizumab variant, comprising administering a Neisseria meningococcal Type B specific vaccine 14±3 days prior to the administration of the eculizumab or an eculizumab variant, or after that period of time but before about 14 days after the first administration of the eculizumab or an eculizumab variant.
